(19) United States
(12) Patent Application Publication
(10) Pub. No.: US 2005/0250141 A1
(43) Pub. Date: Nov. 10, 2005

(54) Diagnostic assays including multiplexed lateral flow immunoassays with quantum dots

(76) Inventors: James L. Lambert, Sunland, CA (US); Anita M. Fisher, La Crescenta, CA (US)

Correspondence Address: GREENLEE WINNER AND SULLIVAN P C, 4875 PEARL EAST CIRCLE, SUITE 200, BOULDER, CO, 80301, US

(21) Appl. No.: 11/094,115

(22) Filed: Mar. 30, 2005

Related U.S. Application Data
(60) Provisional application No. 60/557,540, filed on Mar. 30, 2004.
(60) Provisional application No. 60/583,982, filed on Jun. 30, 2004.

(51) Int. Cl. C12Q 1/68; G01N 33/554; G01N 33/569; C12M 1/34

(52) U.S. Cl. 435/6; 435/7.32; 435/287.2

(57) ABSTRACT

Multiplexed lateral flow assays, related methods, and devices are disclosed which are capable of simultaneously detecting multiple analytes. The assays are preferably immunoassays and can be multiplexed spatially, spectrally, and both spatially and spectrally. Multiplexed assays are disclosed employing quantum dots for applications including the detection of human proteins and the monitoring of microorganisms relevant to water contamination. The invention is widely adaptable to a variety of analytes such as biowarfare agents, human clinical markers, and other substances.

### CROSS-REFERENCES TO RELATED APPLICATIONS

This application claims the benefit of U.S. Provisional Applications 60/557540, filed Mar. 30, 2004; and 60/583982, filed Jun. 30, 2004. Each of these previous applications is incorporated herein by reference in entirety.

### STATEMENT ON FEDERALLY SPONSORED RESEARCH OR DEVELOPMENT

This invention was made, at least in part, with government support under grant/contract NAS7-1407 awarded by the National Aeronautics and Space Administration (NASA). The government may therefore have certain rights in the invention.

### BACKGROUND OF THE INVENTION

Many Lateral Flow Assays (LFAs) are immunoassays that can be used to detect chemical or biological agents in various media including food, water, blood, urine, and saliva. The most commonly used LFA is the home pregnancy test which is performed frequently with minimal training or experience. The home pregnancy test is an example of an immunoassay designed to detect a single compound, human chorionic gonadotropin, from a urine sample. LFAs are also commercially available for use in food and water safety, for detection of Escherichia coli, Salmonella, Legionella, etc.; food processing and food safety, for detection of food allergens such as peanuts, shellfish, etc.; clinical medicine, for detection of hCG, HIV, hepatitis C, etc.; and homeland defense (anthrax, botulinum toxin).

The ability of diagnostic tests based on immunoassay principles to detect a tremendous variety of substances can be attributed to the potential and actual diversity of antibodies. Antibodies to nearly any chemical structure can be developed. For example, a mouse produces 10¹¹ different types of antibodies, many never encountered in the evolutionary lifetime of the animal including the lifespan of the individual; antibodies to explosives such as TNT and RDX are now available for use in immunologically-based detection systems.

In a typical LFA, capillary action draws a sample droplet putatively containing a target molecule, along with tagged antibodies impregnated within a test strip, toward a capture line or zone where specific immobilized antibodies reside. If the target molecule is present in the sample, both the tagged and immobilized antibodies bind to the target, thus forming a complex referred to as a sandwich at the capture line and indicating a positive result. The sandwich of molecules is made up of a first tagged antibody connected to the target and a second antibody also connected to the target. The assay also generally has a control line with nonspecific antibodies. An example of such a nonspecific antibody is a host-specific IgG which will serve as a positive control regardless of whether the host sample has a true positive status on the capture line for a given analyte. Here the control line is designed to capture tagged antibodies that fail to bind to the capture line. The control line can therefore function to confirm that a test is functional or valid independently of whether the capture line indicates a negative or positive result.

Commonly available LFAs utilize tagged antibodies that are labeled with colloidal gold or latex nanospheres. Such labels are used to form colorimetric indicators for reporting whether the target molecule is present. These simple strip tests provide rapid results in a few minutes, are very easy to use in the field, and can be relatively inexpensive. Conventional LFAs, however, are generally only useful as a qualitative diagnostic test. Furthermore, a separate single LFA is required for each chemical or biological agent of interest. For applications such as astronaut health monitoring, homeland security, and other applications, a single assay capable of detecting multiple analytes would be useful. Consolidation into a single assay format offers potential advantages. In the face of the large number of possible biowarfare agents, one advantage is minimizing the time needed to identify and respond to a threat. Other possible advantages are a reduction in necessary materials and cost. The disclosed multiplexed LFAs are suitable for field use in a variety of venues and offer capabilities beyond provide this capability.

The advent of quantum dots, also referred to as nanocrystals and semiconductor nanocrystals (e.g., photostable color-tunable nanoparticles with a wide absorption spectrum and a narrow emission peak), has allowed a fresh opportunity to explore the improvement of immunoassays, including multiplexed immunoassays. Quantum dots (QDs) have high quantum efficiency (on the order of 0.5), resist photobleaching, and can be produced in colloidal suspensions with a narrowband emission spectrum (about 30 nm). During fabrication, the diameter of QDs can be selected to achieve emission fluorescence in a variety of colors. QDs are therefore desirable candidates for use as tags in qualitative or quantitative multiplexed LFAs.

Multiplexed lateral flow assays, including such assays employing quantum dots, could potentially be used to detect many compounds on a single strip. There is a practical difficulty, however, due to effects such as non-specific binding (NSB). Non-specific binding is a phenomenon that occurs between or among different recognition molecules such as antibodies and causes “cross-talk” between tests. As a consequence, multiplexed assays tend to have higher background signals that limit parameters such as dynamic range, sensitivity, specificity, potential for quantitative measurement, and clinical accuracy, e.g. false positives.

There is therefore a need for improvements in the field of diagnostic assays, including multiplexed lateral flow immunoassays, reader apparatus, and related methods of fabricating such assays.

### SUMMARY OF THE INVENTION

The following abbreviations are applicable: QD, quantum dots; LFA, lateral flow assay; QDLFA, quantum dot-based lateral flow assay; ISS, International Space Station; NSB, non-specific binding; N, number of tests multiplexed on an LFA; M, multiple number of lanes on a multiplexed LFA; FRET, fluorescence resonance energy transfer; SavQD; streptavidin quantum dot.

The present inventors believe we are the first group to provide a lateral flow assay using antibodies tagged with quantum dots as fluorescent indicators. The invention provides for spatial and spectral multiplexing of quantum dot lateral flow assays (QDLFA), thereby generating simple strip tests that can measure the levels of several to many chemical or biological agents concurrently. In an embodiment, the tests employ fluorescence detection to allow quantitative measurement of levels of these agents.

The invention provides a method of fabricating multiplexed immunoassays wherein the immunoassays are capable of high sensitivity for detection of an analyte.

In an embodiment, the invention provides a method of detecting a plurality of target analytes in a sample containing or suspected of containing the plurality of analytes, comprising the steps of: (a) providing the sample on a solid support; (b) providing a plurality of conjugates wherein each conjugate is specific for each target analyte, and wherein each conjugate is a semiconductor nanocrystal conjugate having an emission spectrum distinct from the other conjugates; (c) combining said sample with said conjugates, wherein said combining is performed under conditions that allow formation of complexes of each specific conjugate and each specific target analyte, when present; (d) removing any unbound conjugate; (e) spatially arranging a plurality of capture zones wherein each capture zone has a capture reagent specific to said target analytes; and (f) detecting at said plurality of capture zones the presence of said complexes, if present, by monitoring a spectral emission mediated by the semiconductor nanocrystal in said complexes, wherein the emission indicates the presence of one or more target analytes in the sample.

In an embodiment, said solid support can function as a lateral flow assay utilizing capillary action to mediate a fluid flow of said sample. In an embodiment, said each conjugate that is specific for each target analyte is an antigen recognition molecule. In an embodiment, the sample is a water sample. In an embodiment, the sample is a human clinical sample. In an embodiment, the sample is a material suspected of exposure to a bioterrorism event. In an embodiment, an analyte is a microorganism, protein, polysaccharide, drug, or nucleic acid molecule.

In an embodiment, an effect of a non-specific binding contribution on an asay of said method is reduced to a level comparable to an effect of a non-specific binding contribution on an assay for a single analyte.

In an embodiment, the invention provides a method for spectrally encoding a spatially multiplexed lateral flow assay, comprising: defining a detection reagent set of Z detection reagents, wherein Z equals at least two; creating a plurality of unique spectral profiles from one or more fluorophore reagents; and assigning said spectral profiles to said detection reagents, wherein each detection reagent from 1 to Z receives a unique spectral profile. In an embodiment, Z is from 2 to about 100. In an embodiment, Z is from 2 to about 10.

In an embodiment, a spectral profile of said spectral profiles relates to a fluorophore reagent or combination of fluorophores capable of exhibiting a unique spectral emission peak. In an embodiment, said fluorophore or combination of fluorophores utilize at least one type of semiconductor nanocrystal. In an embodiment, a spectral profile is generated from a bead conjugate incorporating a combination of fluorophores chosen to produce a spectral emission which is orthogonal to a spectral emission of another bead conjugate. In an embodiment, said assay is an immunoassay in a lateral flow assay format.

In an embodiment, said assay is a water monitoring assay. In an embodiment, said assay is capable of detecting a plurality of agents selected from the group consisting of E. coli, Streptococcus group A, Pseudomonas aeruginosa, Staphylococcus aureus, and Stenotrophomonas maltophilia.

In an embodiment, the invention provides a reader apparatus for reading an output of a lateral flow assay, comprising: (a) a fluorescence spectrometer; (b) a securing means for holding a sample strip so as to allow the strip to be responsive to said spectrometer; and (c) a translocatable positioner capable of effecting a displacement of said strip with respect to said spectrometer.

In an embodiment, said differential positioning is with respect to an x or y axis of said strip. In an embodiment, said differential positioning is with respect to an x and y axis of said strip.

In an embodiment, said lateral flow assay is a spatially multiplexed lateral flow assay. In an embodiment, said lateral flow assay is a spectrally multiplexed lateral flow assay. In an embodiment, said lateral flow assay is a spatially and spectrally multiplexed lateral flow assay.

In an embodiment, the invention provides a method for reading a spectrally encoded, spatially multiplexed assay comprising: (a) providing a reader apparatus; (b) providing an assay solid phase matrix subsequent to assay initiation; (c) exposing said matrix to said apparatus so as to allow a first measurement at a first matrix position corresponding to a capture zone of said assay; (d) measuring at said first matrix position; (e) translocating said strip relative to said reader; (f) exposing said matrix to said apparatus so as to allow a second measurement at a second matrix position; and (g) measuring at said second matrix position. In an embodiment, said at least one of said first or second measurements measures a spectrally filtered emission signal.

In an embodiment, the invention provides assays in kit form.

In an embodiment, the invention provides a quantum dot-based lateral flow assay (QD-LFA). In an embodiment, a QD-LFA uses fluorescent quantum dots to create LFAs that combine increased sensitivity with the added advantages of quantitative testing and multiplexing capability. In a particular embodiment, a QD-LFA is adapted for pregnancy testing. In a particular embodiment, a QD-LFA is a spectrally multiplexed assay.

In an embodiment, a multiplexed LFA is achieved by spatial separation of a plurality of capture zones. In another embodiment, a multiplexed LFA is achieved by such spatial separation in combination with spectrally encoding a set of recognition molecules specific for a respective set of analytes.

The invention provides multiplexed lateral flow assays and methods of making multiplexed LFAs that mitigate a background signal produced by non-specific binding (NSB) between or among the multiplexed testing reagents. In an embodiment, the background of multiplexed LFAs can be reduced to levels seen in single-agent LFAs. In an embodiment, the assays and methods are adaptable to other multiplexed immunoassays, biochips, etc.

In an embodiment, a multiplexed LFA is adapted for screening for microbial contamination in a potable water supply. In a particular embodiment, an assay is adapted to screen a potable water supply of the International Space Station.

In an embodiment, the invention provides a method for detecting one or more analytes by producing and reading a spectrally encoded, spatially multiplexed assay which reduces non-specific binding to levels seen in single analyte lateral flow assays. In a particular embodiment, one or more detection antibodies corresponding to one or more respective analytes are conjugated with one ore more detectable labels with one or more unique spectral emission peaks. In a preferred embodiment, one ore more of the detectable labels is a quantum dot. In another embodiment, one or more of the detectable labels is a conventional fluorophore. In another embodiment, one or more of the detectable labels is a conventional label as known in the art; e.g. a conventional fluorophore (including fluoroscein isothiocyanate, rhodamine, etc.), gold, latex, magnetic or paramagnetic material, colorimetric reagent, other chromogen, or other tag.

In an embodiment, capture antibodies for each analyte are placed in lines at spatially different locations. For example, the different locations can be distributed at successive points distal from a sample placement point along a single axis, e.g. in a configuration similar to the rungs of a ladder. Alternatively, the different locations can be distributed geometrically in a radial pattern similar to a wheel-and-spoke configuration; where each spoke may optionally employ multiple capture or detection zones at successive points distal from a sample origin point.

The invention provides a reader apparatus for detection of signal from the assay. In an embodiment, the reader apparatus is designed to measure emitted light at one or more physical sites wherein recognition molecules (such as capture antibodies) collect or are affixed. In an embodiment, the apparatus is designed to spectrally filter an emission signal to selectively detect light emitted by the corresponding spectrally encoded detection antibody or other recognition molecule. In an embodiment, the reader apparatus is adapted to read an assay dynamically in real time as the assay develops or statically at a selected time point after initiation of the assay.

In an embodiment, quantum nanoparticles are used as a fluorophore label and a single excitation light source is employed.

In an embodiment, a detectable label is a structure comprising a plurality of different labels. In a particular embodiment, such label is a bead conjugate incorporating a mixture of fluorophores (e.g. quantum nanoparticles) wherein a spectral emission profile of a first bead conjugate is chosen to produce a spectral emission which is distinguishable from (e.g. orthogonal to) at least one other bead used in the in lateral flow assay.

In an embodiment, several tests are multiplexed within a single lateral flow assay. In a particular embodiment, a reader apparatus is designed to spectrally filter for a particular spectral signature assigned to each spatial location of a corresponding recognition molecule such as a capture antibody.

In an embodiment, an assay of the invention is a biosensor for testing a fluid sample. In a particular embodiment, the fluid sample is a physiological fluid such as interstitial fluid, sweat, urine, whole blood, serum, or plasma. In a particular embodiment, the biosensor is capable of continuous or periodic monitoring of the fluid sample. In an embodiment, the biosensor is modified to detect multiple analytes. In a particular embodiment, the biosensor is for multiple analytes for non-invasive monitoring of physiological fluids.

In another embodiment, the fluid sample is from a water storage, water reclamation, or water purification system. In a particular embodiment, the biosensor is adapted to measure multiple analytes relating to microbial contamination in water.

In an embodiment, assays, methods, and devices of the invention are broadly adaptable to detect microorganisms (e.g. bacteria, viruses, fungi, protozoa), including pathogens relevant to potential water contamination, food safety, and clinical disease; environmental safety, biodefense monitoring and biowarfare agents; human and animal clinical markers; drugs; polypeptides; nucleic acid molecules; and other substances.

In a particular embodiment, a multiplexed LFA is capable of measuring two or more species or strains of bacterial simultaneously. In a particular embodiment, a single or multiplexed LFA is capable of quantitative measurement of a level of a bacterium or two or more species of bacteria.

In an embodiment, a lateral flow assay is a dipstick assay.

It is well understood in the art that an immunoassay can be configured in many ways. For example, a configuration can be respective of whether an analyte, antigen, antibody, or other detection or recognition molecule is fixed or mobile, conjugated, or arranged so as to have a positive signal report a binding event or to have a negative signal report a binding event such as in an inhibition assay. Similarly, the use of blocking and washing buffers, for example to reduce non-specific binding to substrates or to wash away unbound reagents at various steps, is well understood in the art and can be implemented in applications. Moreover, the use of various reagents (e.g., biotin and avidin) to expand the possible ways of connecting molecules and potentially amplify output signals is also understood. The present invention is thus intended to encompass many configurations for applications.

It is recognized that regardless of the ultimate correctness of any mechanistic explanation or hypothesis, an embodiment of the invention can nonetheless be operative and useful.

### BRIEF DESCRIPTION OF THE FIGURES

FIG. 1 illustrates the architecture of a basic lateral flow assay.

FIG. 2 illustrates a multiplexed LFA under idealized conditions.

FIG. 3 illustrates results from a completed lateral flow assay under more realistic conditions involving nonspecific binding.

FIG. 4 illustrates the application of spectral filtering to a spatially multiplexed LFA.

FIG. 5 illustrates an example of NSB noise scaling with varying N, where N=2 or N=4.

FIG. 6 illustrates an example of spectral encoding applied to detect human plasma proteins.

FIG. 7 illustrates an example of a spectrally encoded QDLFA for detection of three different bacterial agents: E. coli, Streptococcus group A, and Pseudomonas aeruginosa.

FIG. 8A, FIG. 8B, and FIG. 8C illustrate reader apparatus for use with multiplexed LFA.

FIG. 9A illustrates the relatively narrow emission bands produced by quantum dots. FIG. 9B illustrates that higher level multiplexing is achieved by further variations including the use of multiple sample lanes. FIG. 9C illustrates that a recognition molecule can be spectrally encoded with a distinct profile.

FIG. 10 illustrates results from spectrally multiplexed LFAs by two different antibody schemes to detect human plasma proteins.

FIG. 11 illustrates lateral flow assays for hCG using gold beads in one assay, streptavidin coated quantum dots in another assay, and the linear relationship of quantum dot output intensity versus quantum dot concentration.

FIG. 12 illustrates a Water Test Kit used on the International Space Station which involves a microbial capture device.

FIG. 13 illustrates: (left panel) Fluorescence spectra of ebGFP-eGFP dimer in the presence of 1 mM Ca²⁺ and EDTA; (right panel) Frequency domain lifetime measurements of ebGFP unquenched donor (blue), and ebGFP-eGFP dimer in 1 mM Ca²⁺ and absence of Ca²⁺.

FIG. 14 illustrates plasmid maps relating to FRET sensors.

### DETAILED DESCRIPTION OF THE INVENTION

In general the terms and phrases used herein have their art-recognized meaning, which can be found by reference to standard texts, journal references and contexts known to those skilled in the art. The following definitions are provided to clarify their specific use in the context of the invention.

When used herein, the term “recognition molecule” refers to a material capable of binding with specificity to a target analyte. For example, a recognition molecule can be an antibody (optionally referred to as a capture antibody). Other such materials include aptamers; natural, recombinant, or synthetic fragments of antibodies (including scFv); receptors for ligands or counterreceptors; and derivatives or analogs thereof.

When used herein, the term “spectrally encoded” refers to a reagent that is labeled with a single label or multiple labels so as to provide a distinguishable or coded label set associated with the reagent in comparison to another reagent. For example, a set of three antibodies can be spectrally encoded by labeling each with a single unique quantum dot label. Alternatively, a set of three antibodies are spectrally encoded as follows: a first antibody is labeled with a single unique first quantum dot label; a second antibody is labeled with a combination of the first quantum dot label and a second quantum dot label, and a third antibody is labeled with a combination of a second quantum dot label and a third quantum dot label.

The invention may be further understood by the following non-limiting examples.

### EXAMPLE 1

### Spatially and Spectrally Multiplexed Lateral Flow Assays

We began our work developing LFAs that used antibodies (for several different microbes) tagged with a single color of quantum dot. Only one color quantum dot was available at the time that was both water soluble and functionalized with streptavidin, allowing it to be easily linked with a biotinylated antibody of choice. We initially developed spatially multiplexed LFAs by striping test lines of capture antibody corresponding to each type of microbial strain of interest for testing. We noticed that nonspecific binding frequently occurred. The degree of NSB observed was typically proportional to the number of assays multiplexed (and the number of types of antibodies used) in a given LFA. The presence of NSB, not autofluorescence of the substrate or sample matrix, was the largest background signal contributing to outcomes of limiting sensitivity and specificity of all our tests on the LFA.

Subsequently functionalized quantum dots became available in a variety of colors. We continued to use separate test lines for each agent but now with corresponding antibodies tagged with unique colors. Using this procedure we could see the test results visually without the need to use a spectrophotometer for analysis. We tested our multiplexed LFAs with single strains of microbes initially to measure signal to background levels, etc. When we did this, we noticed that nonspecific binding still occurred on the various test lines, but now it presented in color. Ideally only one test line should present with a given color, therefore all other lines displaying that color are due to NSB that now can be selectively detected or eliminated, for example by optically filtering out a given signal. Since the emission spectra of quantum dots are very narrow, multiplexed LFAs without the background signal contribution from one or more other tests can be produced. This encoding scheme is a useful option in the development of practical multiplexed LFAs.

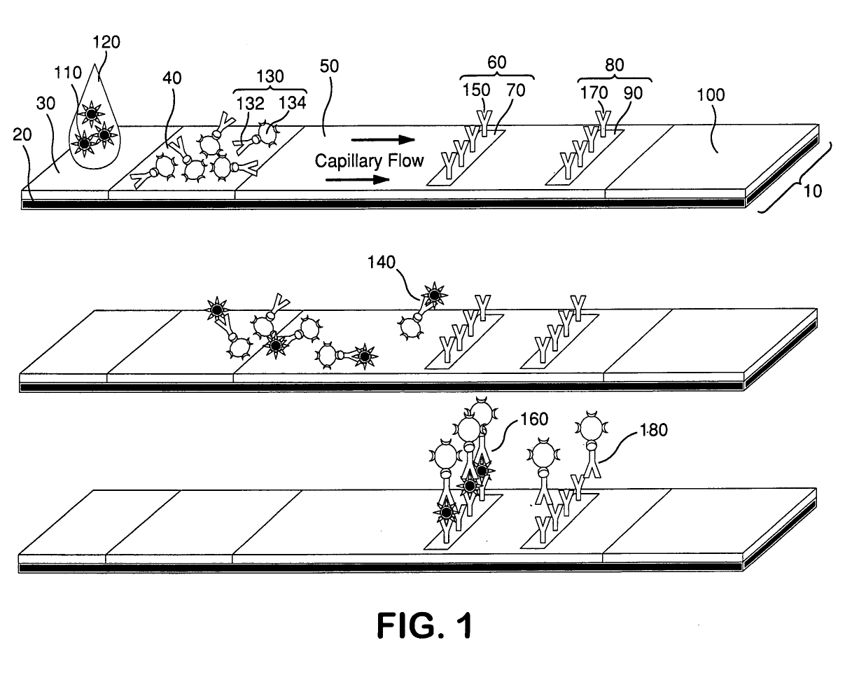

FIG. 1 shows the general architecture of a lateral flow assay (top panel, assay initiation; middle panel, assay in progress with lateral flow from left to right; bottom panel, assay completion). Test strip 10 with backing substrate 20 has a sample pad 30, conjugate pad 40, nitrocellulose membrane component 50, first capture zone 60 with test line 70 and second capture zone 80 with control line 90, and wicking pad 100. Here, analytes 110 in sample droplet 120 are exposed to sample pad 30. The analytes 110 encounter detection antibody conjugates 130 comprised of detection antibodies 132 and tags or labels 134. In some cases the conjugates 130 specifically bind analytes 110 and form first complexes 140; complexes 140 and antibodies 130 are drawn by capillary flow towards capture zones 60 and 80. First complexes 140 encounter capture antibodies 150 and form second complexes 160, in this case indicating a positive result for detection of the analyte. Some antibodies 130 encounter control antibodies 170 and form third complexes 180, confirming the validity of a functional test.

We investigated the possibility of reducing noise from nonspecific binding in multiplexed lateral flow assays. Nonspecific Binding (NSB) is inherent to all lateral flow assays. Without countermeasures such as filtering, noise due to NSB in multiplexed assays is proportional to N, where N is the number of tests multiplexed on the LFA. By spectrally encoding each type of detection antibody using QDs of different colors and spatially multiplexing each type of capture antibody on separate test lines, noise due to NSB can be reduced to similar levels seen in a single agent LFA.

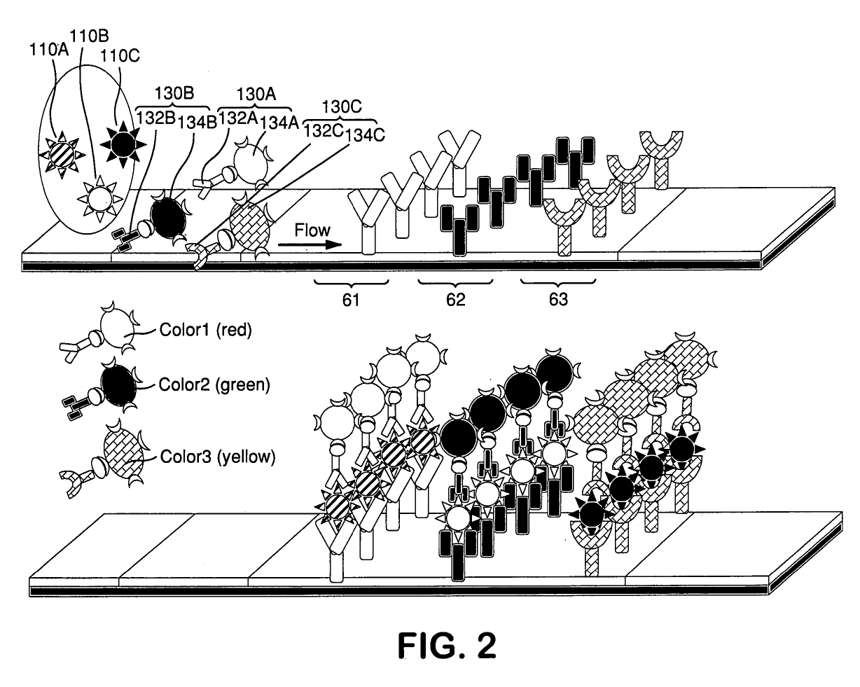

Spatial multiplexing, or the separation of different detection reagents into physically distinct capture zones, can allow the development of an assay for detection of multiple agents. FIG. 2 illustrates how spatial multiplexing can detect three analytes (for example, three different microbial agents of potential relevance for water contamination) under idealized conditions. These conditions assume no contribution from nonspecific binding. Nonspecific binding could arise in various ways: (a) fixed capture antibody 1 binding to the antibody or conjugated label components for any of mobile phase conjugate antibody 1, mobile phase antibody conjugate 2, and mobile phase antibody conjugate 3; and (b) fixed capture antibody 1 binding to the analyte portion of any of mobile phase complex 1 (complex of analyte 1 with conjugate antibody 1), mobile phase complex 2 (complex of analyte 2 with conjugate antibody 2), and mobile phase complex 3 (complex of analyte 3 with conjugate antibody 3). It may be more likely that the contribution of cross-specificity of one antibody type for another different antibody type will affect assay performance than other contributions of nonspecific binding.

FIG. 2 shows an immunoassay during initiation (upper panel) and at completion (lower panel). Three different analytes are subject to detection: first analyte 110A, second analyte 110B, and third analyte 110C. Three corresponding detection antibody conjugates are employed: first conjugate 130A, having antibody 132A and label 134A; second conjugate 130B, having antibody 132B and label 134B; and third conjugate 130C, having antibody 132C and label 134C. The labels here are for exemplary color wavelengths for red (label 134A), green (label 134B), and yellow (label 134C). The multiple capture zones are therefore designed to facilitate detection of the three distinctly coded labels in first capture zone 61, second capture zone 62, and third capture zone 63.

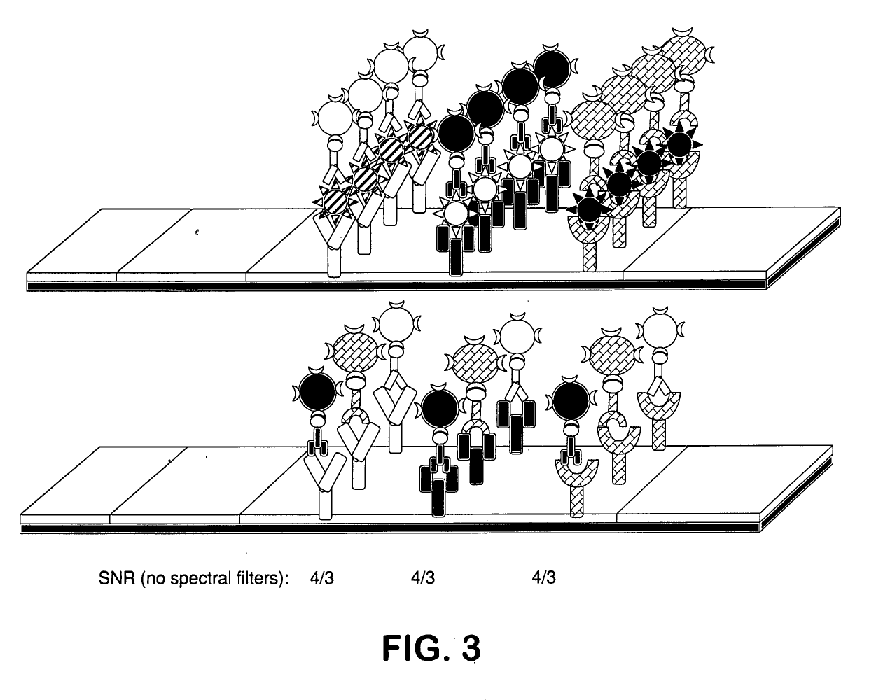

Along with spatial multiplexing of different capture antibodies, however, it is advantageous to use a differential coding scheme for each type of capture antibody. Although the use of coding is depicted in FIG. 2, the coding aspect is not fully exploited. FIG. 3 depicts results from a completed lateral flow assay under more realistic conditions involving nonspecific binding. The reported output signal has components of true signal (upper panel) and noise such as from nonspecific binding (lower panel) and can be shown as an equation: Ouptut=Signal+Noise. Here there is an assumption of 25% NSB among all combinations of detection and capture antibodies. The example illustrates that NSB noise increases proportionally with N, the number of tests multiplexed in the assay. The signal to noise ratio (SNR) without the benefit of spectral filters for each capture zone is 4:3.

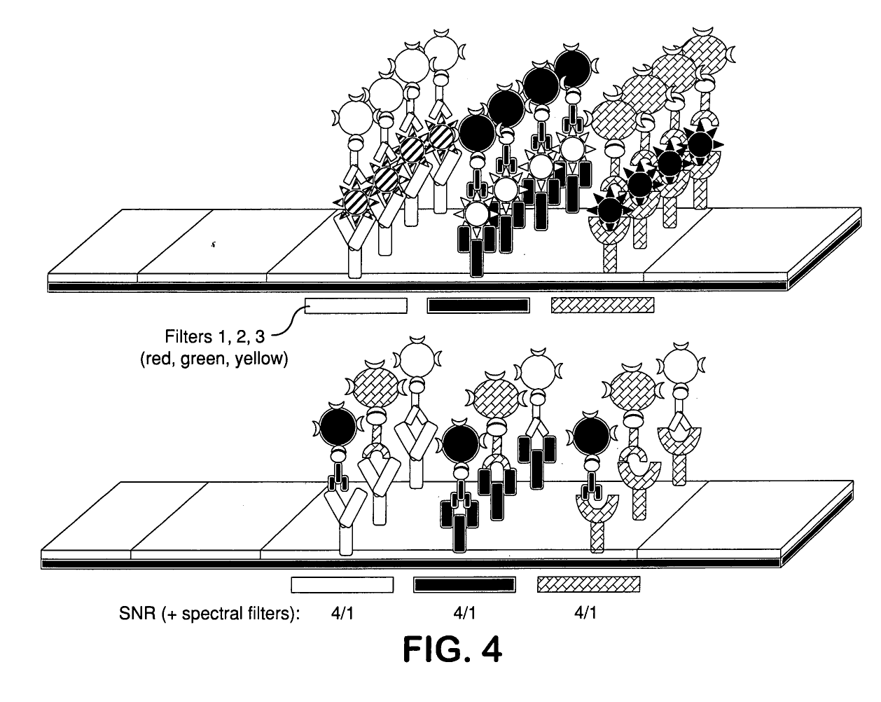

The advance of applying spectral filtering to the spatially multiplexed assay is illustrated in FIG. 4. Again there is an assumption of 25% NSB. Appropriate filters 1, 2, and 3 (e.g. red, green, and yellow) are used to read the total output signal for each capture zone. The signal to noise ratio with the benefit of spectral filtering has a decreased proportion of noise (SNR=4:1) for each capture zone. Significantly, there is now an opportunity to hold NSB noise to a relatively constant level, or minimize the contribution of NSB noise, in multiplexed assays with increasing N.

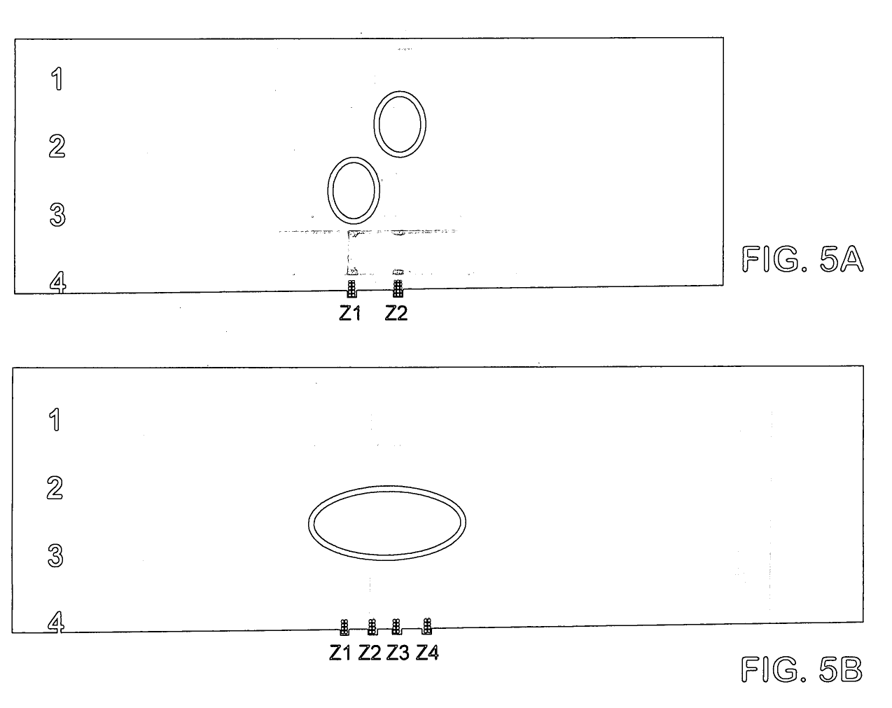

FIG. 5 illustrates an example of NSB noise scaling with varying N, where N=2 or N=4. FIG. 5A shows a 2-agent, one color LFA where the two agents were E. coli and Streptococcus group A (respectively corresponding to capture zones Z1 and Z2). FIG. 5B shows a 4-agent, one color LFA where the four agents were E. coli, Streptococcus Group A, Pseudomonas aeruginosa, and Staphylococcus aureus (respectively corresponding to capture zones Z1, Z2, Z3, and Z4). All runs used roughly 5×10⁶ organisms per type of agent. The circled/highlighted areas indicate the presence of NSB crosstalk. In FIG. 5B, more crosstalk is observed as the number of multiple agents is increased from two to four; such increased crosstalk lowers the effective signal-to-noise ratio. The following test strips were used as shown in Table 1 and Table 2.

TABLE 1
Test strips in FIG. 5A.

| Strip no. | Strip Code | Description |
|---|---|---|
| 1 | 0 | None |
| 2 | EC | E. coli |
| 3 | SGA | Streptococcus group A |
| 4 | EC, SGA | E. coli + Streptococcus group A |

TABLE 2
Test strips in FIG. 5B.

| Strip no. | Strip Code | Description |
|---|---|---|
| 1 | 0 | Water |
| 2 | 0 | Buffer and Blocker |
| 3 | PA | Water |
| 4 | PA | Buffer |

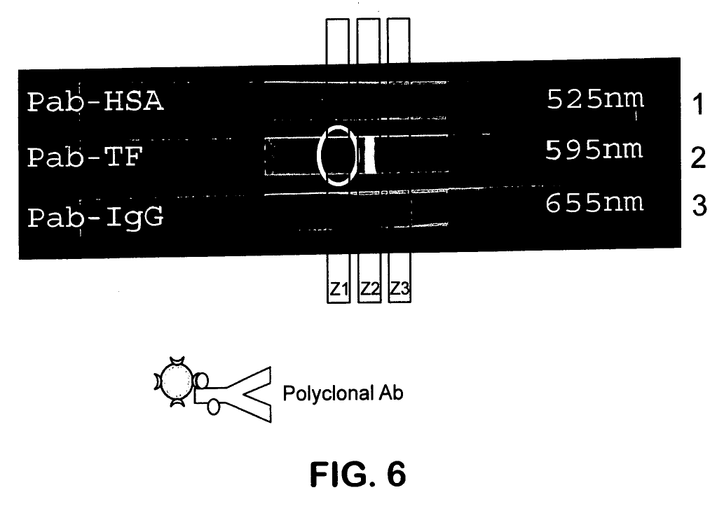

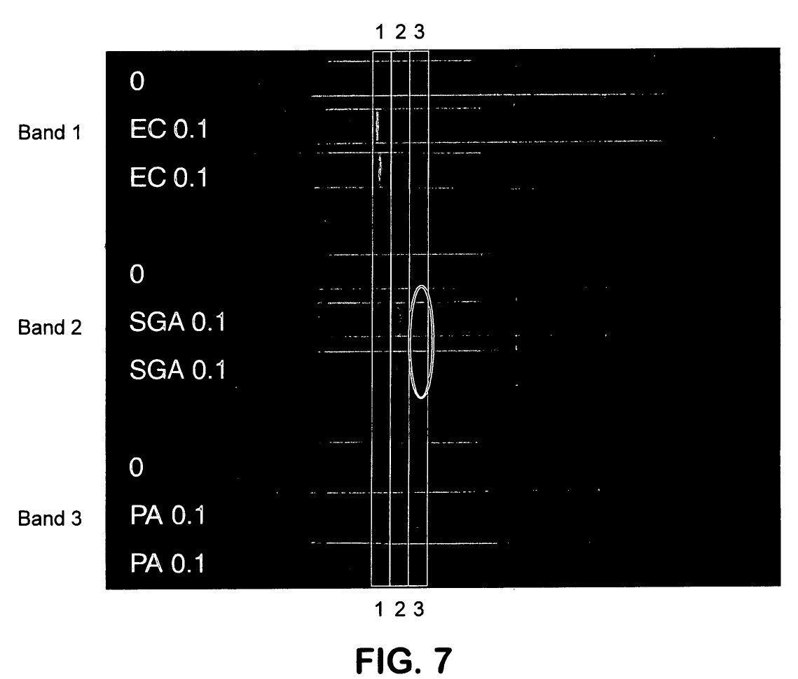

FIG. 6 illustrates an example of spectral encoding applied to detect human plasma proteins. The example utilized primary and secondary antibody conjugation schemes, using polyclonal antibodies (Pab), for on-strip detection of human serum albumin (HSA), transferrin (TF), and immunoglobulin G (IgG). Referring to the circled/highlighted area in the middle strip denoted as Pab-TF, since capture zone 1 (Z1) is assigned a green wavelength, this yellow NSB crosstalk can be rejected by employing a green filter. The detection wavelengths denoted for strips 1, 2, and 3 are 525 nm, 595 nm, and 655 nm respectively.

FIG. 7 illustrates an example of a spectrally encoded QDLFA for detection of three different bacterial agents: E. coli, Streptococcus group A, and Pseudomonas aeruginosa. Three sets of bands are shown. In Band 1, E. coli is detected using polyclonal antibody conjugate Pab-SavQD565. In Band 2, Streptococcus group A is detected using polyclonal antibody conjugate Pab-SavQD605. In Band 3, Pseudomonas aeruginosa is detected using polyclonal antibody conjugate Pab-SavQD655. Each of the antibody conjugates employs a fluorescent quantum dot bound to streptavidin (Qdot™ nanocrystals obtained commercially from Quantum Dot Corporation, Hayward, Calif.). The capture zones or test lines are noted as 1, 2, and 3. Since test line Z3 is assigned the color red at the wavelength of 655 nm, the faint crosstalk marked by the circled/highlighted area at 605 nm (orange) due to NSB can be rejected by employing an appropriate filter such as a red filter.

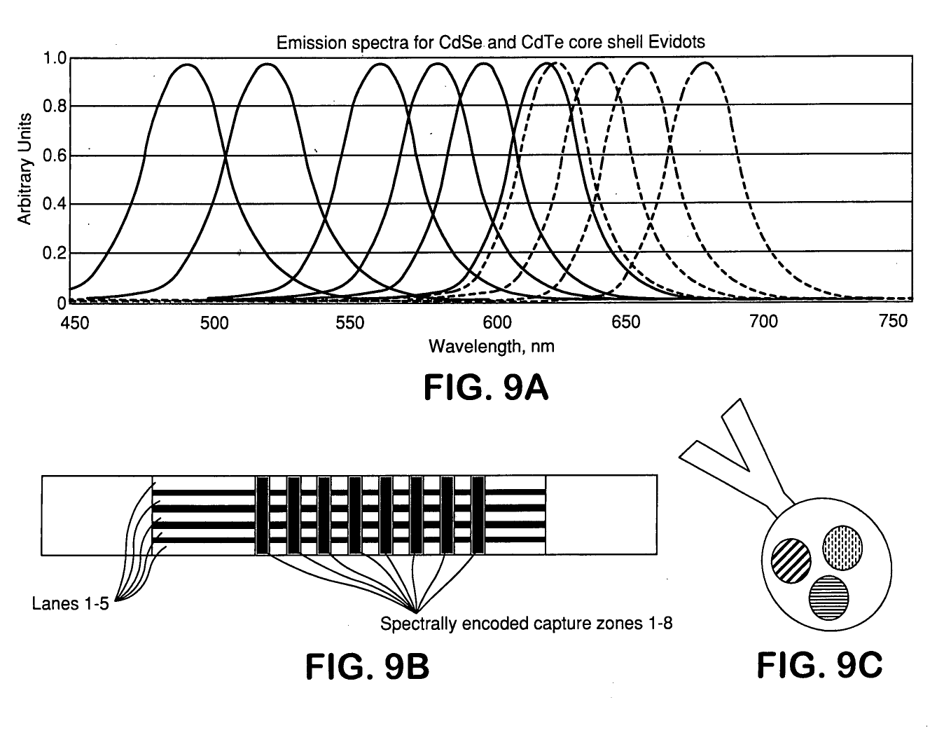

FIG. 9A illustrates the relatively narrow emission bands produced by quantum dots. Shown are various CdSe/ZnS QDs across the visible spectrum allowing multiple agents to be tested per multiplexed LFA. For example, 10 agents may be multiplexed to allow simultaneous testing on one strip. FIG. 9B illustrates that higher level multiplexing is achieved by further variations including the use of multiple sample lanes. For example, a set of multiple flow lanes, M, each with N spectrally multiplexed test lines or capture zones are optionally used to test for a total number of assay substances, T, where T=N×M. A strip is shown allowing for a test of 40 agents (8 spectral codes corresponding to capture zones×5 lanes).

FIG. 9C illustrates that a recognition molecule can be spectrally encoded with a distinct profile. Multicolor beads have been commercially developed containing fixed ratios of several quantum dots. These can be used as spectral bar codes when attached to antibodies or other recognition molecules. For example, rather than encoding each type of antibody with a unique single color based on a single quantum dot or other label, one can assign each antibody a unique spectral barcode, wherein the unique code is selected so as to be distinguishable. In a preferred embodiment the unique codes are chosen so they are mathematically orthogonal from one another. Then many agent LFAs can be realized if one processes the spectra at each test line to determine the number of detection antibodies with the correct code. The contribution of NSB can therefore be minimized or eliminated.

The antibody in FIG. 9C is conjugated to a label where three quantum dots are selected with different colors as summarized in the table below. Further complex profiles can be prepared by having multiple dots per color, etc.

TABLE 3
Example of bar coding to generate a spectral profile.

| | Color | | | | | | |
|---|---|---|---|---|---|---|---|
| | R | O | Y | G | B | I | V |
| | Red | Orange | Yellow | Green | Blue | Indigo | Violet |
| Profile | 1 | 1 | 0 | 0 | 1 | 0 | 1 |
| Value | | | | | | | |

Generally, nonspecific binding currently limits the sensitivity and specificity of LFAs. NSB in a multiplexed LFA scales with N, where N is the number of tests per strip. By designing the multiplexed LFA so that the detection antibody in the presence of its target binds to a unique spatial location, one can spectrally filter out the noise effect of NSB from other detection antibodies used in the multiplexed assay. Multicolor quantum dots or beads may be used to produce distinct spectral codes or profiles, including orthogonal codes, which allow an increase in the number of tests that can be successfully multiplexed.

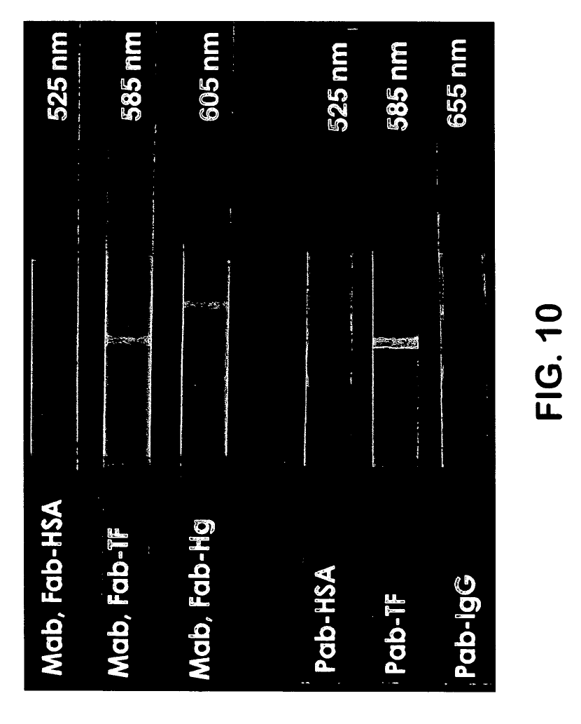

We have developed spectrally multiplexed LFAs by two different antibody schemes to detect human plasma proteins. See FIG. 10. Spectrally multiplexed assays have not previously been possible with conventional detection using gold/latex conjugates for a single color result. Using Qdot Streptavidin Conjugates, each antigen is distinguished by a different color Qdot Conjugate, and several antigens can be tested on one strip. This is made possible because the many colors of quantum dots are capable excitation by the same wavelength segment, unlike organic fluorescent dyes which can often require different excitation sources. We have been successful in multiplex detection of four plasma proteins: human serum albumin (HSA), transferrin (TF), haptoglobin (Hg), and immunoglobulin G (IgG). FIG. 10 shows results from a spectrally multiplexed LFA for plasma proteins, comparing monoclonal F(ab) and polyclonal F(ab) binding to Qdot Conjugates.

Materials and methods. For the top set of three strips in FIG. 10, monoclonal antibodies (Mab) were conjugated to biotinylated anti-mouse F(ab) fragments labeled with Qdot Streptavidin Conjugates provided by Quantum Dot Corporation, Hayward, Calif. For the bottom set of three strips, biotinylated polyclonal antibodies (Pab) were directly conjugated to Qdot Streptavidin Conjugates. Images were captured on a UV transilluminator with a color digital camera.

### Further Materials and Methods

Antibodies, Quantum Dot and Assay Reagents. Whole molecule IgG of mouse, rabbit, goat and sheep were obtained from Jackson Immunoresearch Labs (West Grove, Pa.). These were used as antigens for direct striping on LFA membrane. In non-multiplexed assays of mouse or rabbit IgG, the corresponding anti-lgG (H&L) biotin conjugate used was also from Jackson Immunoresearch Labs. For multiplexed IgG LFA, the corresponding anti-lgG (H&L) antibodies were obtained from Pierce Biotechnology Inc. (Rockford, Ill.), as long chain biotin conjugates and serum-protein absorbed to the other species for minimally cross-reactivity as follows: goat anti-mouse IgG, mouse anti-rabbit IgG, mouse anti-goat, rabbit anti-sheep IgG.

The following infectious disease antibodies were purchased as affinity-purified preparations from BioDesign International (Saco, Me.). Polyclonal antibodies (host goat and biotinylated host rabbit) anti-E.coli and Streptococcus group A, Pseudomonas aeruginosa (host guinea pig) and Staphylococcus aureus (host rabbit). To prepare LFAs for human plasma proteins, purified proteins (the antigens) and both monoclonal (Mab) and polyclonal (Pab) antibodies were obtained from Sigma-Aldrich (St Louis, Mo.), as follows: human immunoglobulin G (IgG), human serum albumin (HSA): mouse anti-HSA clone 11 MAb, rabbit anti-HSA PAb, human transferrin (TF), goat anti-human TF PAb, human haptoglobin (Hg), mouse anti-human Hg clone 36 MAb. An anti-human TF MAb was additionally obtained from BioDesign International. The anti-human IgG was a goat PAb biotin conjugate from Jackson Immunoresearch Labs.

Secondary antibodies (anti-lgG) are widely available biotinylated, as listed above; however, the biotinylated primary antibodies, such as anti-P. aeruginosa and anti-S. aureus, were prepared in our laboratory using the Pierce EZ-Iink NHS-LC-biotin kit according to the recommended reaction protocol for IgG proteins. Briefly, the NHS-biotin and IgG were incubated at a molar ratio of 15:1 on ice for 2 hours. The resulting biotin-antibody conjugates were purified by dialysis using a slide-a-lyzer MWCO 3000 (Pierce Biotechnology Inc) into PBS buffer, pH 7.4 containing 0.05% sodium azide.

The quantum dot (QD) bioconjugates were provided by Quantum Dot Corporation (Hayward, Calif.) as 1 or 2 μM stock solutions for dilution with QDC buffer (50 mM borate buffer containing 2% BSA, pH 8.3). The majority of the assays were developed with a QD605 streptavidin conjugate (Qdot 605 Sav) and additional colors shown in multiplexed assays (Qdot 525 Sav, Qdot 595 Sav, Qdot 655 Sav).

Lateral Flow Assays. LFA nitrocellulose membranes (HF75 and HF90), glass fiber conjugate pads and cellulose wicks were supplied by Millipore OEM (Danvers, Mass.). LFA tests were assembled by laminating the membrane, conjugate pad and wick materials onto GL-187 clear polyester cards (G&L, San Jose, Calif.). Reagents used for preparing LFA (antigens, antibodies and conjugates) were dispensed using a BioDot XYZ3000 Dispensing System (BioDot, Irvine, Calif.). Capture antibodies were generally striped at 1.0 μL/cm, providing a line about 1 mm wide on the nitrocellulose membrane; antibody concentrations used were generally 1.0 mg/ml diluted in PBS buffer containing 3% methanol. Spacing in between multiple “test lines” was 3 mm apart. Binding of antibody protein to membrane was optimized by a 37° C.×30 min drying step. After striping with capture antibody reagent, the assembled LFA test cards (300 mm length) were cut into 5 mm strips, using a BioDot CM4000 Guillotine, providing 60 tests strips (75 mm×5 mm) per card. The LFA strips were stored with desiccant in sealed bags at ambient temperature. LFA samples of 50 μL or 100 μL were applied to the conjugate pad in a compatible buffer, for example, 50 mM borate buffer containing 2% bovine serum albumin (BSA), pH 8.3. As in any immunoassay, the concentration of antibody giving maximum signal requires titration. The optimum ratio of Sav QD to biotinylated antibody (the bioconjugate) was individually determined for each antibody, and in most cases was obtained at a final antibody concentration of about 10 to about 20 μg/mL together with QD concentration of 10 nM. Titration of the Sav QD to biotinylated antibody ratio depended on two factors: maximum signal relative to background fluorescence that is achieved when antibody is maximally labeled with QD (since unlabeled antibody is not detected in assay). Secondly, QD concentration must not exceed antibody or a “bridging effect” occurs where each QD can bind multiple antibodies and cause precipitation. Samples had finished flowing on the LFA strips after 5-10 minutes and were generally air-dry in 30 minutes. Completed dry test strips were stored in folders protected from light at ambient temperature. Under these conditions, test strips retained fluorescent signal over 12 months.

LFA test results were routinely visualized by placing test strips on a UV 365 nm light box (UVP, Upland, Calif.). The optically clear backing allowed transillumination of test strips. Results were documented with a Kodak Professional DCS digital camera and software.

### EXAMPLE 2

A launch into space in October 2000 began the effort of permanent human habitation of the International Space Station (ISS) at an average altitude of 354 kilometers above Earth. The success of various research and exploration missions and the very survival of personnel depend on many factors. As on Earth, water is an important commodity for space projects. Water is an important commodity for drinking, preparation of dehydrated food, and other purposes. Problems that have been encountered relating to water safety in the space station and for water testing generally have provided part of the motivation to make an improved diagnostic testing system. This is an example of how the investment into space projects translates directly into advancements that can also be applied outside of space projects.

Water monitoring on the ISS is currently performed by first concentrating any bio-contaminants present within a 100 mL water test sample using filtration. Growth media in the filter's housing is used to enrich the sample for a 5 day period. A tetrazolium indicator changes color in areas where growth occurs. There are several problems with the present technique: (1) in the case of a positive test, 5 days have elapsed and the crew may already be ill; (2) in the case of a positive test, one has no idea what is growing in the culture; without knowing what type of microbe(s) is involved, treating the water supply or anyone who has become ill is problematic; (3) in the case of a negative test, there is limited useful information; for example, certain microbes are not culturable in R3A growth medium and are therefore undetectable (e.g., Stenotrophomonas maltophilia, formerly Xanthomonas maltophilia, is a species of bacteria that has been identified in 11 of 55 water samples taken from the MIR space station and is not culturable in R3A; this organism has been shown to be pathogenic in immunocomprised patients).

The filtration step used in ISS water testing can concentrate the sample 100×. The new ISS limit on microbial contamination is 5000 CFU. We have developed quantum dot based lateral flow assays that can detect levels of E. coli below this level. A single quantum-dot multiplexed LFA can be used to determine the identity and level of contamination in a 3-10 minute period so that in the case of a positive test the appropriate measures can be taken before one or more crew members become ill. The technique is also readily adaptable for a variety of other applications including commercial food and water monitoring as well as homeland security and defense applications.

We have labeled biotinylated antibodies with avidin coated QDs. When incubated together, the antibodies bind tightly to the dots via the avidin-biotin connection, forming a QD-labeled antibody. Individual QDLFAs have been demonstrated with a microorganism (known concentrations of viable E. coli bacteria), a chemical agent (hCG) and a protein (IgG) using quantum dots of several different colors. Multiplexing of the LFAs is also achieved. Using the avidin-biotin technique, multiplexed assays are performed using a single test strip. The multiplexing can be done at lower levels, e.g. for single assay detection of 2 to 10 different analytes, or at higher levels, even on the order of greater than about 100 different analytes.

Multi-agent immunoassays for monitoring the integrity of the food and water are useful for aiding in maintaining astronaut health during long duration space flight. Similar technology can also be applied for monitoring the dynamics of the ecology within bioreactors. A desirable feature in the design of these devices is that a minimum of crew interaction is required for maintenance or use.

The supporting fluidic control system capable of allowing variations of an immunoassay to function automatically may be an expensive proposition; furthermore, reliability of these systems can be problematic in a microgravity environment. We develop two types of one-step bioassays that require minimal fluidics processing. The first is a lateral flow assay (LFA) that provides single-use testing for the presence of analytes within a fluid sample. LFA are simple immunoassay strip tests that can be read visually and require no active fluidics system since they rely only on capillary action for fluid flow. Fluorescent or paramagnetically tagged antibodies may be incorporated into LFA and provide the ability to quantitate readings with low-level detection. The second type of system utilizes a peristaltic pump for continuous or periodic sampling through a Teflon AF hollow-core optical waveguide in which antibodies are immobilized along the inner diameter. Other antibodies tagged with quantum dots (QD) are bound, with their corresponding antigen, to the immobilized antibodies on the wall of the waveguide. This sandwich structure forms the basis for this competitive multi-analyte assay. UV excitation light from a light emitting diode is guided down the entire length (2 m) of the hollow-core waveguide. Each assay in the waveguide uses quantum dots with a unique spectral emission characteristic allowing multiple assays to be performed as the sample fluid passes through the waveguide. The instrument determines levels of contamination by monitoring the spectral emission emanating from the waveguide itself or the spectral characteristics of the QD-tagged antibodies as they exit the capillary.

In applications, fluorescently tagged antibodies are incorporated into LFA. Fluorescent LFAs can increase sensitivity by two to three orders of magnitude and can provide both the ability to test for multiple biological agents in parallel as well quantitate the levels detected. Baseline data demonstrating yes/no colorimetric assays as well as fluorescent LFAs utilizing antibodies labeled with quantum dots are developed.

Two products are prepared. A first device is a colorimetric LFA, packaged in a cassette in a manner such that it suitable for use in microgravity. The first device is optionally configured to interface with filtration-based concentrator units currently being utilized on the ISS to analyze high purity water and reclaimed water. The second device is the fluorescent LFA and a corresponding reader. Both systems are tested using a variety of water samples including optionally those returned from the ISS.

Water reclamation is one of the basic requirements of a regenerative life-support system and involves the treatment of reclaimed water from condensate and and/or urine, for its conversion to potable and hygiene water. Part of the water treatment process involves the use of biocides (e.g. iodine or silver solutions) that inhibit microbial contamination. Therefore, an essential component of a spacecraft monitoring system for water quality is the confirmation of the efficacy of water sterilization.

For some time, NASA specifications for product water included a spacecraft maximum concentration level (SMCL) of 100 CFU/100 ml for total bacteria and <1 CFU or PFU/100 ml (e.g., a non-detectable level) for total coliform and viruses, respectively [references 4,5]. However, the Russian SMCL specification has been 10,000 CFU/100 mL. Since the water purification systems on the ISS have been developed by Russia, a new standard of 5000 CFU/100 mL for total bacteria has been established. The standard of less than 1 CFU or PFU per 100 mL for total coliform and viruses remains. Coliforms are present in the environment and are enteric bacteria, so water contamination by fecal coliform and E. coli may not be a health threat in itself, but indicates potential contamination or inadequate maintenance of the water systems.

A heterotrophic plate count (HPC) is a common analytical method employed on the ISS to measure the variety of bacteria that can contaminate water. This task of utilizing an HPC in microgravity is the current standard practice for monitoring microbial growth in space. The HPC is labor intensive and is a non-specific test. The fluorescent or colorimetric LFA, on the other hand, can be used for diagnostic screening for specific organisms with high specificity and/or sensitivity. Unlike HPC, LFAs can be used to detect agents that are not readily culturable such as viral pathogens and have been commercially introduced (e.g. HIV, hepatitis LFA test kits).

We develop spectral multiplexing provided by fluorescence-based LFAs and an accompanying reader apparatus. Spectral multiplexing allows the capability of screening for multiple pathogens in parallel with increased sensitivity at about two to three orders of magnitude. One key benefit of the increased sensitivity is a concomitant reduction in the water sample volume required for concentration prior to using the test. Therefore, the LFAs we develop can provide valuable diagnostic information to the ISS crew. Multiplexed LFAs can have potential advantages of saving precious crew-time and replacing or augmenting more traditional screening methods including non-specific screens such as HPC.

We develop immunosensors that are suitable for use in microgravity and meet the size, weight, and sensitivity requirements required for microbial monitoring of potable water on the ISS. In conventional, ground-based diagnostic applications, enzyme immunoassay and to a lesser extent, immunofluorescence, are widely used [6] but they are generally performed in fully equipped microbiology laboratories, rather than under “field” conditions. These methods based on monoclonal antibody technology offer specificity through the ability to distinguish the antigen of interest from other antigens. Adsorption of antigens and antibodies in solid phase formats (including nitrocellulose membrane) can broadly differentiate the genus or detect the species or specific antigenic serotype of the microorganism. More than one microbial species can be detected in a sample if the specific antibodies are labeled with different tags such as fluorophores, providing the possibility of multiplexed measurement. Sensitivity of these assays can be enhanced utilizing a preenrichment step. Assay enhancement is achieved using fluorogenic and chemiluminescent substrates, combined with electrochemical or magnetic detectors, and also by flow immunofiltration assay [7,8].

Routine detection methods for testing the potability of water and detecting the presence of bacteria generally involve concentration via membrane filtration, in which water samples are filtered through a device and microorganisms collect on a polycarbonate membrane [7]. Following this, the membrane is removed aseptically, and the sample is transferred into a non-differential medium for incubation. Testing by culture of viable bacteria and standard plate count takes 48 hrs to 5 days. However, microbial monitoring requirements are not restricted to any specific unit system, such as colony forming units (CFU); in fact, dependence on growth of microorganisms should be avoided or reduced. Monitoring can involve detection of cells, cell remnants or cell markers. Many detection methods are now based on molecular biological techniques which can have advantages such as accuracy and sensitivity [6]. However, these methods are highly specialized and are therefore less suitable for implementation by space crew in microgravity environments.

The lateral flow assay (LFA) format we develop is based on a non-competitive antigen assay, comparable in construction to commercially available lateral flow assays found in the local drug store. We further develop LFAs, however, for microbial contaminants that are not available commercially. A recent ground-based study of MIR water samples taken from samples collected over 4 years indicate the presence of a variety of flora for which no commercial LFA tests exists.

We also develop a fluorescence based LFA which is used to measure the levels of multiple organisms in parallel by employing novel semiconductor nanocrystals, called quantum dots, as fluorescent reporter molecules. These quantum dots have been conjugated to immunoglobulin proteins. The technology we utilize combines antibody specificity, fluorescent label sensitivity, and in particular the ability to multiplex the detection. We develop versatile, minimum maintenance immunoassays for rapid and simple measurement of bacterial contaminants in process water as a rapid test, i.e., results read in minutes which can be repeated each day.

Lateral Flow Assay Components. Buffers used for LFA depend on the analyte being measured, and they frequently contain a variety of constituents suitable for assay performance including serum proteins (e.g. albumins), detergents, surfactants and polymers. One or more of these constituents are used as blocking agents to prevent non-specific binding and background.

Nitrocellulose (NC) membrane is the preferred analytical membrane for LFA. It can be attached to a substrate, for example by lamination to polyvinyl or polyester backing using a pressure-sensitive adhesive that is LFA-compatible, and represents the solid phase matrix for the assay. Specifically, a large pore membrane is chosen that ranges in pore size (e.g., from 5 μM to 20 μM) and protein binding capacity, variables which affect sensitivity, accuracy and lateral flow rate. Sample and conjugate release materials vary in properties and typically require pre-treatment with blocking agents and sample buffer.

The instrumentation for LFA generally requires a precision dispensing platform for quantitative “striping” of the capture and control lines of antibody solutions onto the NC membrane and for striping a detection-antibody conjugate onto the conjugate pad. The Biojet Quanti system (BioDot, Inc., Irvine, Calif.) is an instrument suitable for manufacturing purposes which performs both line and dot applications of reagents. It uses a combination of positive displacement syringe pumps and solenoid valves to provide a non-contact programmable volume of reagent. An accompanying instrument for fabrication of test strips is the BioDot Guillotine cutting system, also controlled by a hand-held programmable device.

Detection Reagents often conventionally used for LFA include the following reporter molecules: colloidal gold, latex beads (various colors), and colloidal paramagnetic particles (non-visible signal, but provides quantitation). The conjugation chemistries and characteristic properties vary for each of these. Colloidal gold and latex microspheres are the standard reporter molecules used in commercial diagnostic LFA. We have used 40 nm colloidal gold in preliminary assay development; it has a red-purple signal output. Its conjugation via passive adsorption has suitable reliability and requires relatively lower protein amounts. Latex (200-400 nm) microspheres generally require more antibody for the covalent conjugation, although latex allows for a choice of colors which is employed in some diagnostic tests to suit the sample type. Both of these reporter molecules are less suitable for visual detection of very weak lines, due to low dye intensity.

Quantitation of LFA has mostly been achieved to date by using colloidal paramagnetic particles (PMP) as the reporter, ranging in magnetite content and size of 100-300 nm. In order to achieve a quantitative result, a dedicated magnetic assay reader (MAR) or a magnetic susceptometer is required.

Enhanced LFA using Quantum Dot Detection. Quantum dots (QD) are nanocrystals, including semiconductor nanocrystals, with superior fluorescent properties. Luminescent quantum dots can offer an enhanced detection system for LFA of microorganisms and their toxins. To describe the photophysical properties of quantum dots, when a semiconductor absorbs a photon having an energy greater than its bandgap, an electron is promoted from the valence band into the conduction band leaving behind a positively charged hole. The electron-hole pair is called an exciton. Excitons are like artificial atoms having radii of 1 to 10 nm depending on the properties of the semiconductor. As the size of the semiconductor crystal becomes similar to the size of the exciton, strong quantum confinement modifies the exciton properties. With decreasing crystal size, the exciton behaves more like a particle-in-a-box. Its energy levels are determined largely by the size of the particle (box) instead of the properties of the bulk semiconductor. Semiconductor nanocrystals that exhibit strong quantum confinement in all three dimensions are called quantum dots [9].

Recombination of the electron and the hole produces light. The wavelength of light is largely determined by the size of the quantum dot. Quantum dots of extremely uniform size can be made and have a narrow emission bandwidth in the range of 10 to 50 nm. The emission wavelength can be shifted hundreds of nanometers by simply changing the quantum dot size. Excitons can interact with defects in the solid that reduce the energy of the emitted photon. Excitons can also oxidize or reduce adsorbates instead of emitting photons. One way to minimize these undesired exciton relaxation pathways is to coat the surface of the quantum dot with a shell of a higher bandgap material. Coated quantum dots that we have tested have quantum efficiencies of around 55 percent. The coating also protects the quantum dot from its micro-environment. Protected quantum dots are very resistant to photobleaching; typically the rates are 100 or more times lower than those for organic dye molecules [10].

Quantum dots have broad absorption bands that extend well into the ultraviolet. Their emission wavelength is essentially independent of the excitation wavelength, so quantum dots having narrow bandwidth emission at wavelengths throughout the visible spectrum can be excited by a single excitation wavelength, wavelength segment of spectrum, or source. The luminescent lifetimes of quantum dots tend to be in the range of 30 to 100 nanoseconds [11]. This is much longer than background fluorescence and Raman scattering of most sample matrices. We therefore can use time-gated detection to selectively reduce or remove background fluorescence. Since detection limits are often determined by background and not sensitivity, assays based on time-gated detection of quantum dot luminescence are capable of extraordinarily low detection limits.

Synthesis of Quantum Dots and Conjugation to Streptavidin. Antibodies cannot easily be directly conjugated to quantum dots, but using streptavidin as a bridge is a simple way to link biotinylated antibodies and form a stable QD conjugate. Orange-red quantum dots (peak 605 nm) have been used in our initial immunoassay development. These are “core/shell” semiconductor nanocrystals comprised of a CdSe core and a ZnS shell. Preparation of a conjugated quantum dot involves additional capping to attach the streptavidin molecule. The quantum yield of the QD conjugate is 0.55 and the emission FWHM (at 605 nm) is 27 nm. This emission wavelength is conveniently visualized by fluorescence microscopy with FITC, Cy-3, or Alexa-568 filter sets. The 1 μM solution of “conjugated quantum dots” (calculated to be one streptavidin molecule per quantum dot crystal) is biologically similar to about 60 μg/ml of streptavidin alone. The protein-conjugated nanocrystals are stabilized in water and biological buffers at about pH>7. The precise size of streptavidin-quantum dots is unknown but calculated to be about 10 to 15 nm final size. This represents an order of magnitude smaller than paramagnetic particles and four times smaller than colloidal gold.

The binding of multivalent avidin such as streptavidin to biotin is one of the strongest known binding pair interactions. Biotin-labeled antibodies, specifically biotinylated secondary antibodies are widely available as a common reagent for immunoassays such as ELISA. Although a limited selection of biotinylated primary antibodies are available from antibody suppliers, the biotinylation reaction is a relatively simple procedure and the reagents are widely available and used to label monoclonal and polyclonal IgG (Pierce).

The method for biotinylation of detection antibody (Mab hCG anti-beta, BioDesign Int. #E92850M) was as follows: A molar ratio of 15 for IgG (at a concentration of 1 mg/ml) to Biotin was used. NHS-LC-Biotin was dissolved in DMSO and added at a concentration <10% to antibody in reaction buffer. Incubation was carried out on ice for 2 hours. Following this, free biotin was removed by dialysis against phosphate buffered saline for 18 hours. The amount of LC-biotin incorporation was 3-5 molecules per IgG.

Mouse immunoglobulin (whole molecule) and (biotinylated) goat anti-mouse immunoglobulin is an exemplary binding pair, used in immunoassay development. A series of experiments was set up to evaluate a QD-lateral flow assay. The mouse IgG was striped on NC membrane as a capture protein at varying concentrations over a 3 log range. Different molar ratios of SAvQD (streptavidin-quantum dot) and biotinylated anti-mouse IgG were tested and detection was optimized at a ratio of 1:6.6. Using these parameters, 25 ng mIgG was detectable by LFA.

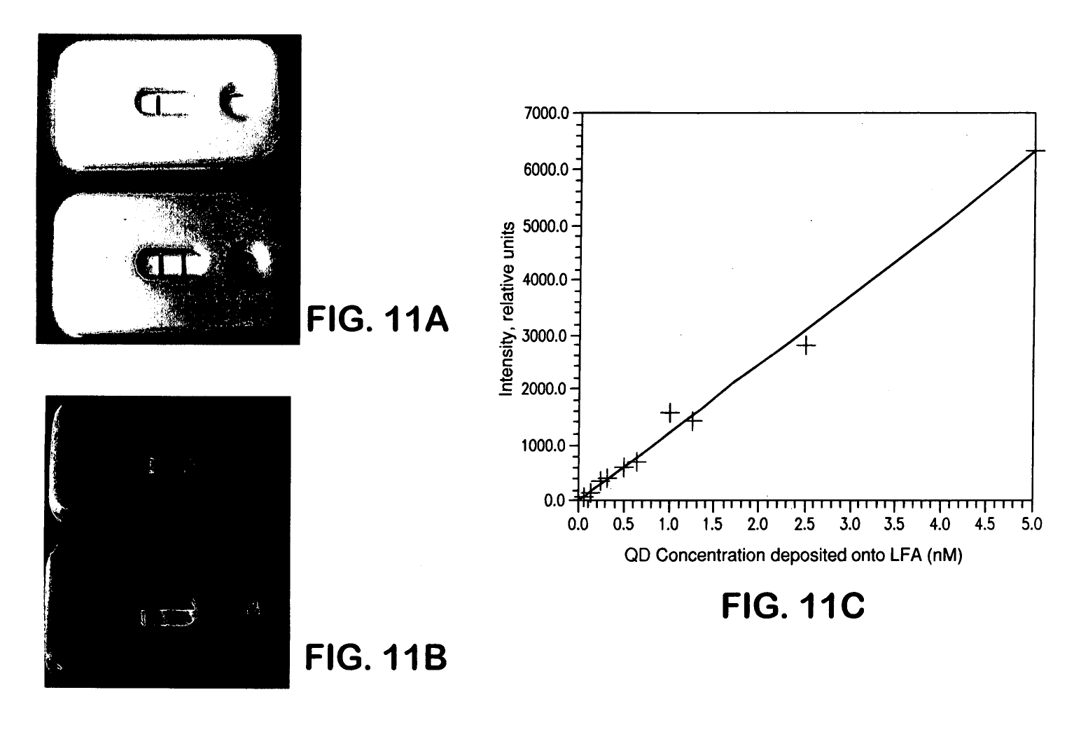

Calibration experiments have been performed to test signal linearity of strepavidin-conjugated QDs. Nitrocellulose membranes (S&S AE98; 5 μM pore; Schleicher and Schuell) were striped with doubling dilutions of stock quantum dot conjugate solution, for the settings commonly used for striping capture antibody (1 μL/cm). Fluorimetry measurements were made with a Kaiser optical system Raman microscope and 10× objective lens. Due to non-fading, high signal intensities, the Kr ion laser was set at 30 μW×0.1 sec (3 μJ). The limit of sensitivity was 312.5 femtomolar quantum dots, with a linear response (R2=0.99) for the range tested (FIG. 11, panel C).

QD-LFA for human hCG was evaluated next since this assay has been used as a model for previous LFA development. The protein hCG is a small analyte. We selected certain antibody pairs for sandwich immunoassay and a known conjugation procedure for colloidal gold. All assay conditions are substantially identical except for the use of biotinylated detection antibody to the conjugate. Test concentrations of 10 to 1000 mlU (milli-international units) hCG are used. Visualization of the fluorescent bands is performed by 354 nm irradiation. FIG. 11 illustrates lateral flow assays for hCG using gold beads in one assay, streptavidin coated quantum dots in another assay, and the relationship of quantum dot output intensity with concentration (panel A, colorimetric gold hCG assay; panel B, fluorescent hCG assay using 603 nm streptavidin coated quantum dots; and panel C, fluorescence intensity of quantum dots striped onto a LFA membrane as a function of concentration. Negative and positive test strips for colloidal gold-LFA (panel A) and streptavidin QD-LFA (panel B) are presented in a commercial format.

Microorganism LFA. Standard isolates have been obtained from ATCC for evaluating detection and limits of sensitivity for the microorganism LFAs: Escherichia coli O157 ATCC#43888, Escherichia coli O125 ATCC#12808 (gram negative rod organisms), and Streptococcus pyogenes, Group A ATCC#8669 (a gram-positive organism). E. coli is the prototype for optimizing immunoassays since a large selection of strains and their corresponding antibodies exist. E. coli O157:H7 is the most studied enteric bacterial pathogen; the toxin-free organism is used in our laboratory. Some serotypes are also a common cause of urinary tract infections (such as E. coli O125:B15). We obtained distinct monoclonal antibodies for these two E. coli serotypes as well as a polyclonal antibody (recognizing all O and K antigens). A monoclonal-polyclonal pair is also used for the Streptococcus Group A sandwich immunoassay, and the two antibodies are interchangeable for capture and detection; both conjugate well with colloidal gold.

An endotoxin (LPS) LFA is also developed, since lipopolysaccharide antigens can persist after a water purification treatment process even though no viable organisms remain.

An advantage of using quantum dots in lateral flow assays is that each individual agent of multiple microbial contaminants (bacteria, viruses, and strains and substrains thereof is distinctly detected in a multiplex assay; this is a major enhancement in the state of the art. We show that multiplexed LFAs are developed with quantum dots by exploiting their photophysical properties, common excitation parameters, extinction coefficients, narrow emission band widths, lack of photobleaching and large color repertoire not found with other fluorophores.

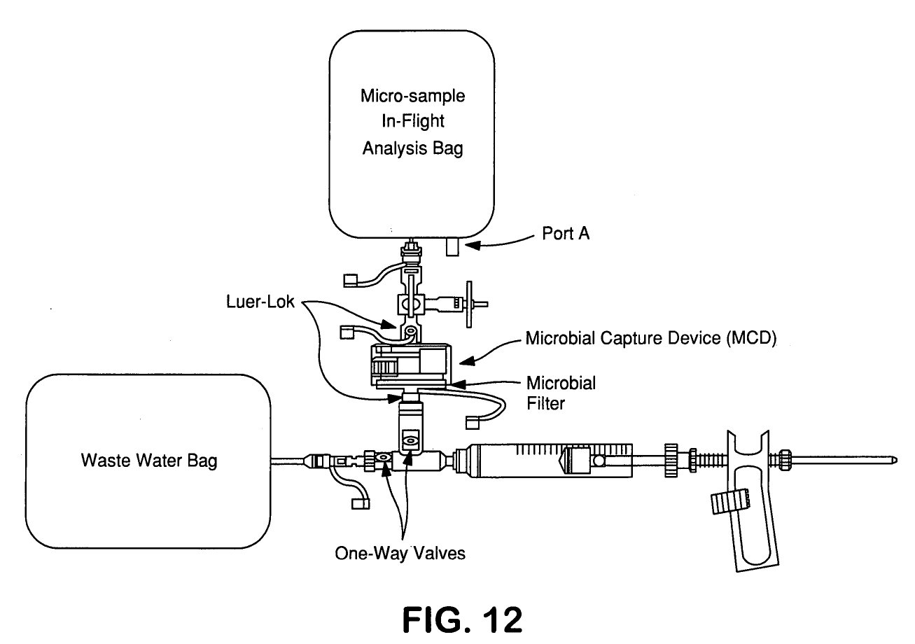

Sample capture devices. Water sampling on the international space station is performed by the crew periodically using a water monitoring kit (WMK) system (FIG. 12). FIG. 12 illustrates a Water Test Kit used on the International Space Station which involves a microbial capture device. The crew member connects port A of the kit to the water source to be monitored (galley port, SVO-ZV, etc). Test water then fills the in-flight analysis bag with approximately 100 mL of the sample. The astronaut then withdraws the syringe which produces a vacuum, drawing a portion of the sample through a filter within the Microbial Capture Device (MCD) as shown. Any bio-contaminants or bio-remnants are captured on the filter. The crew member then pushes the plunger of the syringe to force the filtrate into the large waste water bag. A check valve prevents the filtrate from re-entering the MCD from the bottom. The procedure is repeated until 100 mL sample has passed through the MCD. The MCD device contains R3A media and a colorimetric viability indicator (tetrazolium) which turns purple as a culture grows within the MCD. After incubation, the crew member compares the filter with a chart and records the level of heterotrophic bacterial contamination cultured on the MCD. The MCD is connected with Luer-loks to the WMK as shown and is placed in a biohazard bag after use.

This approach for water monitoring, while effective in providing a general indication of microbial contamination, does not specifically identify any pathogen. Detection of >1 CFU of coliform bacteria would be of concern to the crew since it may indicate contamination with other pathogens.

We develop a “smart” MCD that allows identification of infectious organisms. Lateral flow assays are integrated into cassettes compatible with the WTK (water test kit) used on station. The tests are studied with and without enrichment to measure the presence of specific bacterial species. Two types of WTK-compatible lateral flow assay devices are developed with customization for usage in the space application context: (1) A visually read, colorimetric lateral flow assay and (2) a fluorescent lateral flow assay supplied with an electronic reader. Both are packaged in a MCD-like sealed cassette containing the test device and the sample fluid. The LFA cassettes interface to the WTK using Luer-lok ports provided.

The sample collection procedure is substantially similar to that used with the current water monitoring system. The colorimetric MCD-LFA is read visually. The fluorescence MCD-LFA is subjected to a custom designed reader that is compatible with detecting and indicating the presence or absence of one or more of multiple agents such as multiple strains of bacteria. Sample concentration and/or media enrichment techniques are optionally included depending on a preference for a given assay performance parameter, e.g. a more immediate reporting time versus assay sensitivity.

Four model systems of bacterial strains are used to develop the multiplexed LFA technology: Escherichia coli (O157:H7, ATCC#43888; O125 ATCC#12808), Streptococcus pyogenes (ATCC#8669), and Xanthomonas maltophilia (ATCC#12714). Polyclonal and monoclonal antibodies for each strain are commercially available. S. pyogenes is a representative gram-positive bacterium. Strains of E. Coli were selected because archived data on MIR indicates that these bacteria (and other fecal coliforms) were present on different surface sites throughout the water system [13].

In a study (unpublished) by Duane Pierson and Mark Ott of NASA-Johnson Space Center (JSC), data collected during ground-based studies of 55 different water samples collected from MIR collected from 1995-1998 were tested for culturable bacteria. These indicate that no coliform bacteria survive the water purification process. However, 44 other strains of bacteria were cultured. X. maltophilia was the most common species found, present in 11 of 55 samples cultured. X. maltophilia has been shown to be opportunistic, particularly in people who are immunocompromised [14]. X. maltophilia is of interest because there is a reasonable likelihood that the strain will be present, it represents a minor hazard to the crew, and it appears to be resistant to the normal sterilization procedures used on ISS.

Multiplexed LFA tests for two different serotypes of the model coliform are developed using colorimetric and fluorescent assays. Spatial multiplexing is implemented by placing two different test lines in two locations along the LFA. Colorimetric spectral multiplexing is accomplished by utilizing two different colors of latex beads. Spectral multiplexing with quantum dots is performed by utilizing a dual-antigen serotype LFA utilizing quantum dots of two different sizes with different spectral emission characteristics. Various parameters (e.g. sensitivity, cross-reactivity, etc.) are measured.

Quantitative fluorescent LFAs are developed to provide quantitative information on antigen concentrations using known concentrations of antigens derived from bacterial strains for which the LFAs were designed. Cross-reactivity of the two serotypes of E. Coli is examined and the specificity of the LFAs is evaluated.

### EXAMPLE 3

### Reader Apparatus

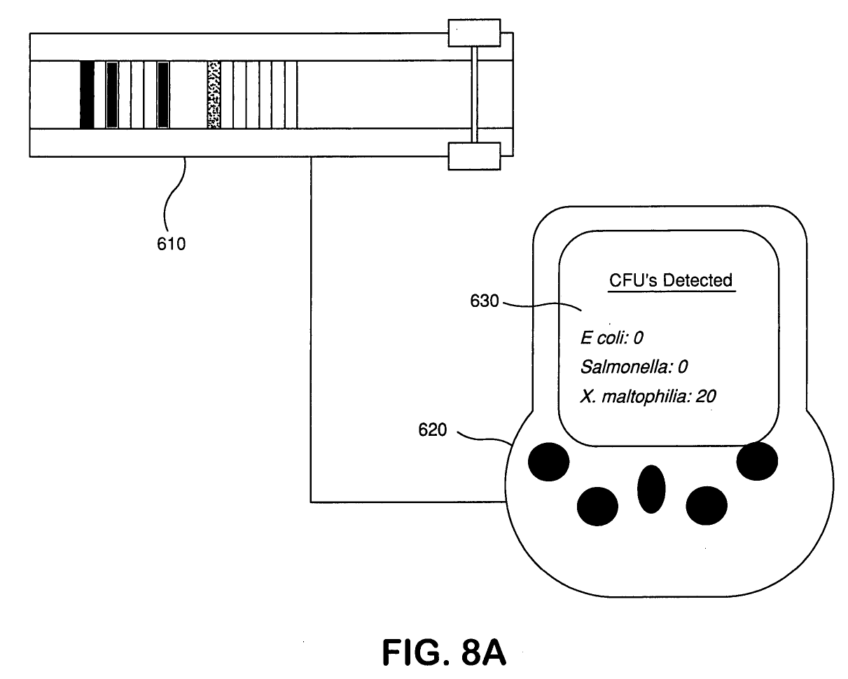

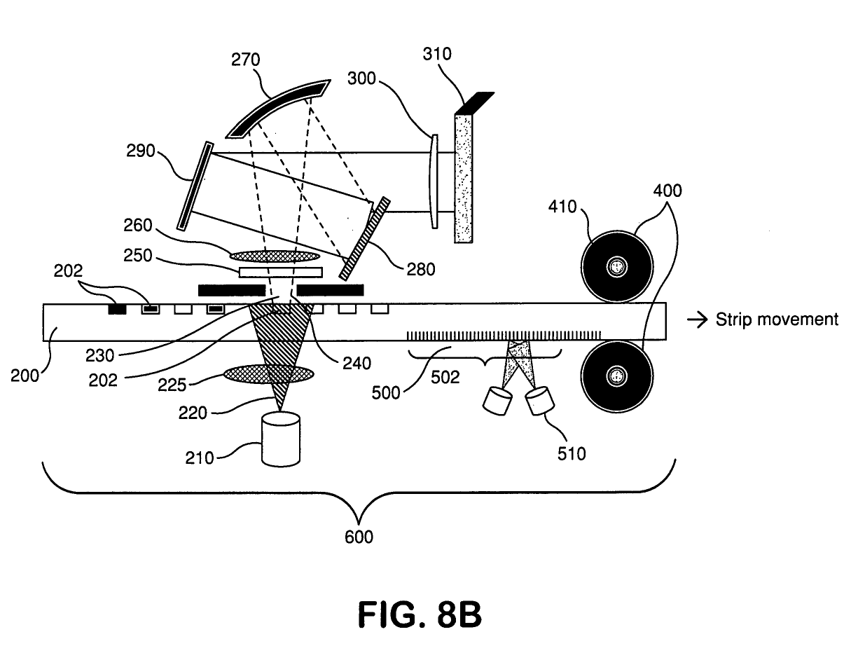

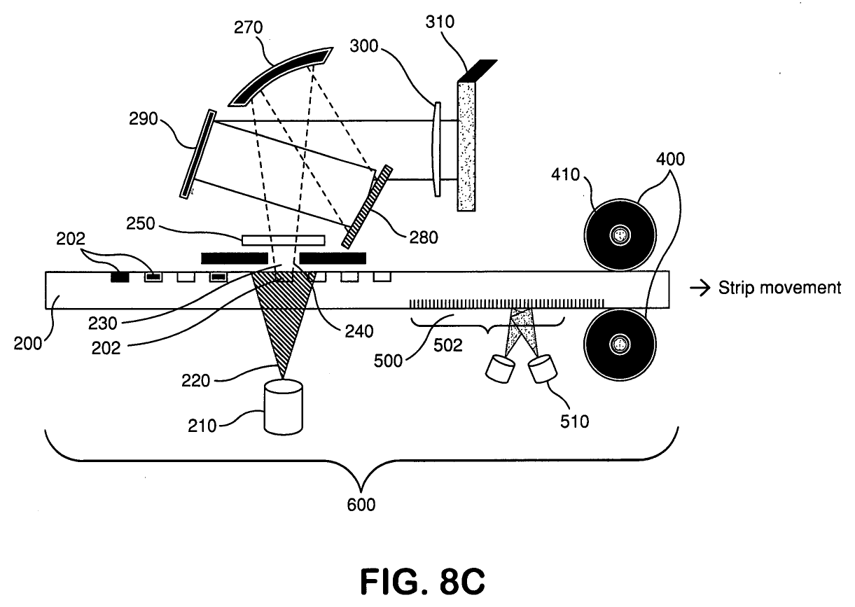

FIG. 8A and FIG. 8B illustrate a reader apparatus for use with multiplexed LFA, including spatially and spectrally multiplexed LFA. A reader is equipped to collect and optionally analyze emission data from assays that are spatially multiplexed or spatially multiplexed and spectrally encoded.

In FIG. 8A, a top view of a strip on a reader mechanism 610 is shown. The reader is optionally connected to a processor such as a computer 620 or other data processing means and output reporter such as a computer display 630, printer, or other reporting means as known in the art. Here, a computer display screen reports output data values for three potential pathogens relating to a water contamination assay.

In FIG. 8B, optical assemblies and mechanical components are shown for a reader apparatus 600. An assay strip substrate 200 is disposed so as to allow excitation source 210 (e.g., an ultraviolet light emitting diode) to transmit an excitation signal 220 optionally through an excitation filter 225 (or filter wheel) towards one or more assay capture zones 202. Calibration lines are optionally included to allow automated or manual orientation regarding the status of initiation, measurement, or completion of data collection for an assay capture zone or assay capture zone set. Excitation light 220 passes through said capture zone 202 producing an emission signal 230. Emission signal 230 passes through aperture 240 and is optionally subject to ultraviolet blocking filter 250 and further optionally subject to emission filter 260. Filters 250 and 260 may optionally be integrated. Filter 260 may optionally be a bandpass filter, selectable wavelength filter, or filter wheel, etc. Emission signal 230 is reflected from first mirror 270 towards grating 280 then reflected from second mirror 290 towards lens 300 and line array detector 310. One of ordinary skill will appreciate that a variety of optical configurations and positionings are understood in the art.

The strip 200 is connected on at least one strip end to a strip feed mechanism 400 which can continuously or periodically provide translation of the strip where the strip translation can occur with respect to the excitation source so as to expose one or more capture zones to such excitation source. Strip feed mechanism is equipped with one or more rollers 410, teeth, adhesive, or other frictional or grasping means for contacting the strip. In the case of rollers 410, the rollers can achieve the contacting or grasping function while simultaneously achieving the translational function. Strip feed mechanism 400 is operatively connected to a manual contact surface or motor and power source (not shown).

The strip 200 is optionally identified with an identification means 500 (e.g. a bar code, alphanumeric designation, or other symbol(s)) in an identification zone 502. The reader is optionally integrated with a reflective optosensor 510 capable of communication, including optical or electronic communication, with the identification means 500.

In a particular reader, a violet LED is used to induce fluorescence of LFAs. In a particular reader, a simple fluorescence spectrometer measures the emission characteristics of the LFAs and a software program interprets the output data. In a particular reader, a computer such as a modern personal computer is used as the controller to interface with an optical reader head.

### EXAMPLE 4

### Fluorescence Resonance Energy Transfer Sensors

Green fluorescent protein (GFP) is a widely used fluorophore in molecular biology applications, useful in part because the sequence and thus, photophysical properties are readily modified. A variety of GFP mutants are available that span the visible spectrum in fluorescence. Taking advantage of fluorescence resonance energy transfer (FRET), which is the transfer of energy from an excited donor to an acceptor chromophore, these different color GFP mutants linked by calmodulin have been demonstrated to be effective Ca²⁺ sensors [1]. We explore the GFP-linker-GFP dimer motif as a general sensing method, in which the calmodulin binding protein linker is changed to the dopamine receptor binding domain. We examine results of FRET distance measurements for firstly a coumarin fluorescein FRET model, then as a function of calcium ion concentration, and subsequently the design of generic GFP-GFP dimers for other sensing applications.

The model used to describe FRET is Förster theory. To test our ability to accurately determine Förster distances we have investigated coumarin as the donor and fluorescein as the acceptor in basic ethanol solution at 10⁻⁵ M. The underlying principle of Förster theory is based on transition dipole-dipole interactions between the excited donor and acceptor. This interaction has a distance dependence of 1/R⁶, where R is the distance between the donor and acceptor. Thus, the efficiency (E) of energy transfer, which is defined as the fraction of excited donor molecules that return to the ground state via energy transfer, equals: E=1/(1+R⁶/Rₒ); where Rₒ is termed the Förster distance, which is the distance at which 50% of the energy is transferred. Rₒ is related to the photophysical properties of the donor and acceptor. After obtaining the fluorescence spectrum of coumarin and the absorbance spectrum of fluorescein, we obtained the Förster distance by employing literature values for the donor fluorescence quantum yield [2] and acceptor maximum extinction coefficient. The so-obtained Förster distance of 5.9 nm is in favorable agreement with published values [2].

Calcium measurements using GFP-FRET. In this investigation, we measured Ca²⁺ concentrations with an all-protein donor-acceptor system consisting of calmodulin (Ca²⁺ binder) fused to flanking enhanced blue GFP (ebGFP=donor) and enhanced GFP (eGFP=acceptor). Specific binding of Ca²⁺ to the central calmodulin linkage decreases the distance between ebGFP and eGFP, thus leading to increased quenching of ebGFP fluorescence due to energy transfer. To evaluate the Förster Distance for the ebGFP-CaM-eGFP dimer, literature values for the donor fluorescence quantum yield and acceptor maximum extinction coefficient [3] were used to determine Rₒ=5.1 nm. The most rapid change in distance occurs between 3.5 and 8 nm. Thus, a sensor based on FRET should exhibit a change in distance within that range upon binding of the analyte for maximum sensitivity. The change in distance between ebGFP and eGFP moieties upon Ca²⁺ binding to calmodulin is approximately 4 to 2 nm.

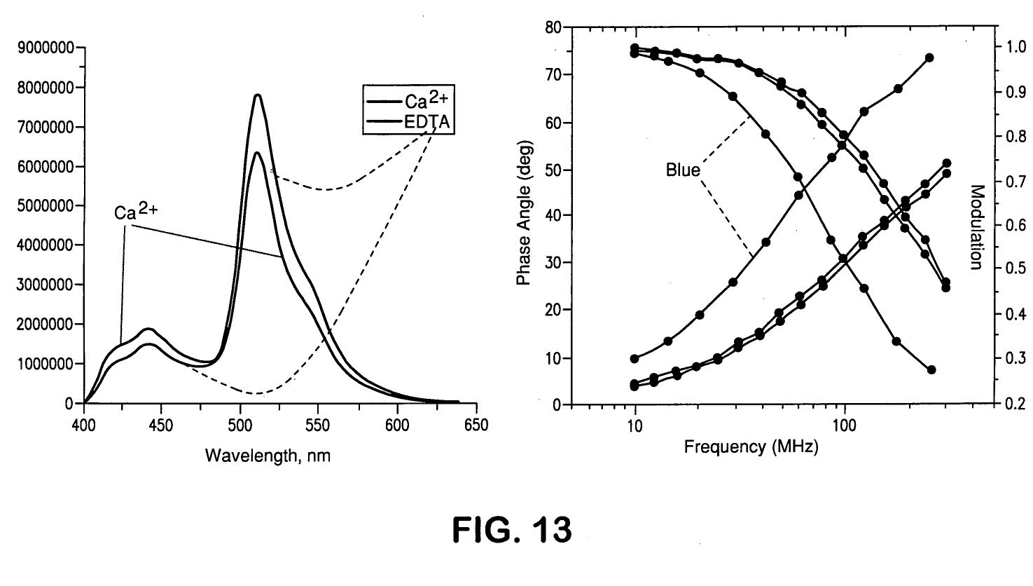

GFP Dimer Distance Distributions. For a FRET sensor like the ebGFP-CaM-eGFP dimer to work optimally, the change in distance upon Ca²⁺ binding should be on the order of 1-2 nm and centered around the Foerster distance, Rₒ. This will lead to the largest signal change when Ca²⁺ is present. FIG. 13 (left panel) shows that the fluorescence of ebGFP donor moiety, centered at 445 nm, increases in the presence of Ca²⁺ while the eGFP acceptor moiety, centered at 515 nm, decreases. While the ratio of fluorescence intensities is certainly measurable, a 10% change in fluorescence intensity indicates small changes in donor-acceptor distances upon Ca²⁺ binding.

FIG. 13 (right panel) shows the frequency domain lifetime measurements of the ebGFP (blue lines which cross towards the left of the plot) and the ebGFPCaM-eGFP dimer (black) in the presence and absence of Ca²⁺. Addition of the eGFP acceptor moiety gives rise to high efficiency energy transfer quenching. However, addition of Ca²⁺ to ebGFP-CaM-eGFP dimer samples does not give rise to measurable changes in signal and thus distance distribution. Clearly, this limits the sensitivity of this system. Nonetheless, the sensitivity is high enough to monitor physiological concentrations of calcium ions. According to frequency domain lifetime measurements, the distance distribution between donor and acceptor GFP moieties does not significantly change upon Ca²⁺ addition. This small change in distance accounts for the small changes in fluorescence intensity in Ca²⁺ titration experiments. This ultimately limits the sensitivity of the ebGFP-CaM-eGFP dimer motif. We concluded that other FRET sensing schemes in which distances and thus energy transfer rates change significantly are useful.

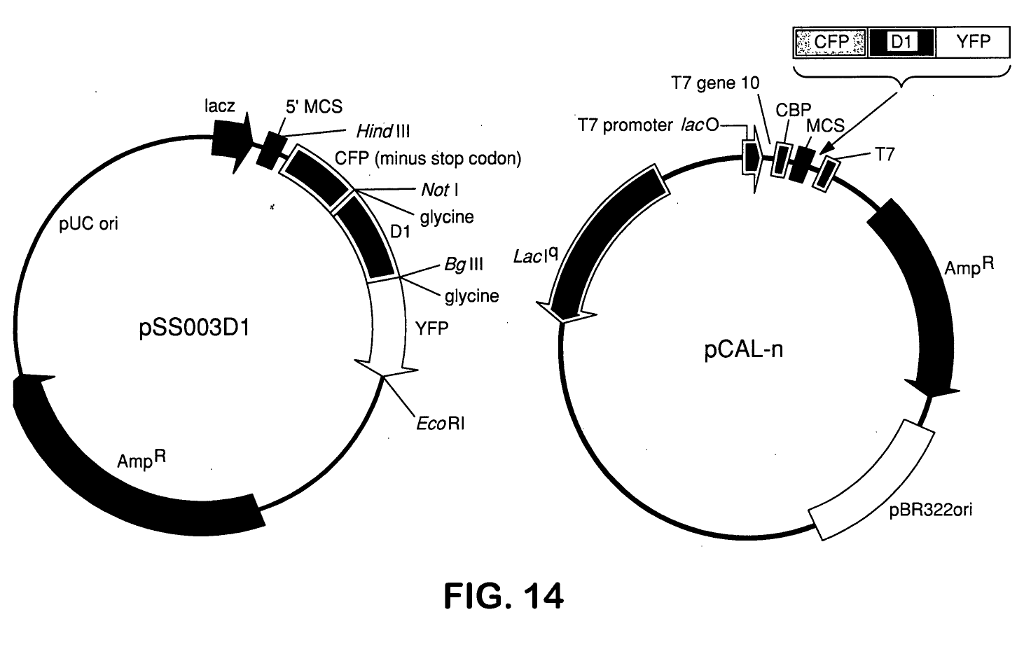

Novel DNA-FRET sensors. In order to adapt the calcium-calmodulin GFP-FRET sensor to detection of other analytes, the calmodulin linker sequence must be replaced, and we chose to insert a receptor binding domain (Dopamine D1/D2) (FIG. 14). This dopamine receptor sensor binds dopamine or could be used for promethazine detection, a drug that is used to treat space motion sickness (SMS). We used variants of the GFP including an enhanced cyan fluorescent protein (eCFP) in combination with an enhanced yellow fluorescent protein (eYFP). These variants were chosen since they are readily available, and easy to clone. The entire gene is subcloned into an expression plasmid so that protein can be produced and purified from bacterial cells. When the inserted construct is expressed in the pCAL-n vector, it is linked to a calmodulin-binding protein (CBP). This allows the inserted protein to be readily purified by affinity chromatography with calmodulin affinity resin (Stratagene). The CBP can be later cleaved away to provide pure protein.

In FIG. 14, the left panel shows a map of plasmid pSS003D1. When an analyte binds to the linker (which in the case of pSS003D1 is the dopamine D1 extracellular binding domain), its length changes. Fluorescent resonant energy transfer (FRET) between the fluorophores is thereby altered, resulting in a ratiometric change in the emission characteristics of the fluorophores. Restriction sites NotI and BglII adjacent to blue (CFP) and yellow (YFP) fluorescent protein sequences allow one to subclone new variants from this plasmid in order to realize new types of FRET chemical sensors. In the right panel, the shown D1 binding domain has an affinity for the drug promethezine. The entire gene including the CFP-D1-YFP is being subcloned into a Stratagene (TM) pCAL plasmid so that the gene may be expressed and purified from bacteria.

Several vectors are generated which link various combinations of eGFPs together as potential sensors. Each of these vectors is tested for viability as FRET sensors by inserting generic linkers of various lengths to test the FRET of their corresponding proteins. Proteins are characterized as viable sensors by analyzing Forster distances and fluorescence lifetime measurements. The generic linkers can be readily replaced with similar specific linkers through simple cloning steps. The resulting “library” of sensor building blocks could be rapidly utilized in conjunction with existing and new protein regions with identified specific binding capabilities.

In applications, the DNA for an array of FRET protein-based sensors each with a different binding linker are synthesized and selectively expressed with appropriate promoters or other regulatory sequences. The constructs are therefore capable of allowing sensors to be selectively expressed in a controlled or even choreographed manner.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Separate embodiments of the invention are also intended to be encompassed wherein the terms “comprising” or “comprise(s)” or “comprised” are optionally replaced with the terms, analogous in grammar, e.g.; “consisting/consist(s)” or “consisting essentially of/consist(s) essentially of to thereby describe further embodiments that are not necessarily coextensive.

The invention has been described with reference to various specific and preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be applied to the practice of the invention as broadly disclosed herein without resort to undue experimentation. All art-known functional equivalents of compositions, methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example or illustration and not of limitation. The scope of the invention shall be limited only by the granted claims.

### REFERENCES

U.S. Pat. No.: 6,500,622 by Bruchez, Jr. et al., issued Dec. 31, 2002; U.S. Pat. No. 6,207,229 by Bawendi et al., issued Mar. 27, 2001; U.S. Pat. Nos. 6,576,291; 6,602,932; 6,607,829; 6,274,323; 6,630,307; 5,990,479; 6,207,392; 6,225,198; 6,306,736; 6,423,551; 6,251,303; 6,306,610; 6,322,901; 6,319,426; 6,326,144; 6,426,513; 6,444,143; 6,602,671; 6,607,829; 6,617,583; 6468808.

U.S. Patent Application Publications: 20020004246 by Daniels, Robert H. et al., Jan. 10, 2002; 20020025529 by Quake, Stephen; et al., Feb. 28, 2002; 20020090650 by Empedocles, Stephen A.; et al., Jul. 11, 2002; 20030143580 by Straus, Don, Jul. 31, 2003; 20030170613 by Straus, Don, Sep. 11, 2003; 20010034034 by Bruchez, Marcel P. et al., Oct. 25, 2001; 20040166505 by Bruchez, Marcel P. et al., Aug. 26, 2004; 20040184954 by Guo, Huiyan, et al., Sep. 23, 2004; 20030008414; 20030148544; 20040260183; 20040127778; 20040019283; 20010034478.

International Patent Publications: WO0029617; WO03003015; WO05001889.

Fisher A M, S. J. Sirk, J. L. Lambert, M. P. Bruchez. Development of a Qdot™ Lateral Flow Assay, A Simple Multiplexing Strip Test; in Quantum Dot Vision, October 2003.

Han Mingyong et al., 2001, Quantum-dot-tagged microbeads for multiplexed optical coding of biomolecules; Nature Biotechnology 19:631-635.

Chemla Y. R. et al., 2000, Ultrasensitive magnetic biosensor for homogeneous immunoassay, Proc Natl Acad Sci USA. Dec. 19, 2000; 97(26): 14268-14272.

Miyawaki A, Llopis J, Helm R, McCaffery J M, Adams J A, Ikura M, Tsien R Y. (1997) Fluorescent indicators for Ca2+ based on green fluorescent proteins and calmodulin. Nature 388, 882-7

Goodall D M, Roberts D R. (1985) Energy-transfer between dyes—a study using fluorescence quenching and Forster Theory. Journal of Chemical Education 62, 711-714

Patterson G H, Knobel S M, Sharif W D, Kain S R, Piston D W. (1997) Use of the green fluorescent protein and its mutants in quantitative fluorescence microscopy. Biophysical Journal 73, 2782-2790

Joint U.S./Russian Potable and Hygiene water specifications for ISS, from P Mudgett and R Sauer

HEDS independent assurance assessment report; evaluation of long-term ISS potable water quality risks, prepared by L K Fotedar, P Brown, D Pate

J E Herrmann. Immunoassays for the diagnosis of infectious diseases, in Manual of Clinical Microbiology, 7th Edition (1999) ASM Press, Washington DC

Ivnitski D, Abdel-Hamid I, Atanasov P, Wilkins E. (1999) Biosensors for detection of pathogenic bacteria. Biosensors and Bioelectronics 14, 599-624

Abdel-Hamid I, lvnitski D, Atanasov P, Wilkins E. (1999) Highly sensitive flow-injection immunoassay system for rapid detection of bacteria. Analytica Chimica Acta 399, 99-108

Gerion D, Pinaud F, Williams S C, Parak W J, Zanchet D, Weiss S, Alivisatos A P. (2001) Synthesis and properties of biocompatible water-soluble silica-coated CdSe/ZnS semiconductor quantum dots. Journal Physical Chemistry B 105, 8861

Chan W C, Nie S. (1998) Quantum dot bioconjugates for ultrasensitive nonisotopic detection. Science 281, 2016

Murphy C J, Coffer J L. (2002) Quantum Dots: A Primer. Applied Spectroscopy 56, 16A

U.S. EPA 816-F-02-013 National Primary Drinking Water Regulations

Rutala W A, Weber D J. (1997) Water as a reservoir of nosocomial pathogens. Infection Control Hospital Epidemiology 18, 1-91

Crispino M, Boccia M C, Bagattini M, Villari P, Triassi M, Zarrilli R. (2002) Molecular epidemiology of Stenotrophomonas maltophilia in a university hospital. Journal Hospital Infections 52, 88

1. A method of detecting a plurality of target analytes in a sample containing or suspected of containing the plurality of analytes, comprising the steps of: (a) providing the sample on a solid support; (b) providing a plurality of conjugates wherein each conjugate is specific for each target analyte, and wherein each conjugate is a semiconductor nanocrystal conjugate having an emission spectrum distinct from the other conjugates; (c) combining said sample with said conjugates, wherein said combining is performed under conditions that allow formation of complexes of each specific conjugate and each specific target analyte, when present; (d) removing any unbound conjugate; (e) spatially arranging a plurality of capture zones wherein each capture zone has a capture reagent specific to said target analytes; and (f detecting at said plurality of capture zones the presence of said complexes, if present, by monitoring a spectral emission mediated by the semiconductor nanocrystal in said complexes, wherein the emission indicates the presence of one or more target analytes in the sample.

2. The method of claim 1 wherein said solid support can function as a lateral flow assay utilizing capillary action to mediate a fluid flow of said sample.

3. The method of claim 1 wherein said each conjugate that is specific for each target analyte is an antigen recognition molecule.

4. The method of claim 1 wherein the sample is a water sample.

5. The method of claim 1 wherein the sample is a human clinical sample.

6. The method of claim 1 wherein the sample is a material suspected of exposure to a bioterrorism event.

7. The method of claim 1 wherein an analyte is a microorganism, protein, polysaccharide, drug, or nucleic acid molecule.

8. The method of claim 1 wherein an effect of a non-specific binding contribution on an asay of said method is reduced to a level comparable to an effect of a non-specific binding contribution on an assay for a single analyte.

9. A method for spectrally encoding a spatially multiplexed lateral flow assay, comprising: defining a detection reagent set of Z detection reagents, wherein Z equals at least two; creating a plurality of unique spectral profiles from one or more fluorophore reagents; and assigning said spectral profiles to said detection reagents, wherein each detection reagent from 1 to Z receives a unique spectral profile.

10. The method of claim 9 wherein Z is from 2 to about 100.

11. The method of claim 9 wherein Z is from 2 to about 10.

12. The method of claim 9 wherein a spectral profile of said spectral profiles relates to a fluorophore reagent or combination of fluorophores capable of exhibiting a unique spectral emission peak.

13. The method of claim 9 wherein said fluorophore or combination of fluorophores utilize at least one type of semiconductor nanocrystal.

14. The method of claim 9 wherein a spectral profile is generated from a bead conjugate incorporating a combination of fluorophores chosen to produce a spectral emission which is orthogonal to a spectral emission of another bead conjugate.

15. The method of claim 9 wherein said assay is an immunoassay in a lateral flow assay format.

16. The method of claim 9 wherein said assay is a water monitoring assay.

17. The method of claim 9 wherein said assay is capable of detecting a plurality of agents selected from the group consisting of E. coli, Streptococcus group A, Pseudomonas aeruginosa, Staphylococcus aureus, and Stenotrophomonas maltophilia.

18. A reader apparatus for reading an output of a lateral flow assay, comprising: (a) a fluorescence spectrometer; (b) a securing means for holding a sample strip so as to allow the strip to be responsive to said spectrometer; and (c) a translocatable positioner capable of effecting a displacement of said strip with respect to said spectrometer.

19. The reader apparatus of claim 18 wherein said differential positioning is with respect to an x or y axis of said strip.

20. The reader apparatus of claim 18 wherein said differential positioning is with respect to an x and y axis of said strip.

21. The reader apparatus of claim 18 wherein said lateral flow assay is a spatially multiplexed lateral flow assay.

22. The reader apparatus of claim 18 wherein said lateral flow assay is a spectrally multiplexed lateral flow assay.

23. The reader apparatus of claim 18 wherein said lateral flow assay is a spatially and spectrally multiplexed lateral flow assay.

24. A method for reading a spectrally encoded, spatially multiplexed assay comprising: (a) providing a reader apparatus; (b) providing an assay solid phase matrix subsequent to assay initiation; (c) exposing said matrix to said apparatus so as to allow a first measurement at a first matrix position corresponding to a capture zone of said assay; (d) measuring at said first matrix position; (e) translocating said strip relative to said reader; (f exposing said matrix to said apparatus so as to allow a second measurement at a second matrix position; and
(g) measuring at said second matrix position.

25. The method of claim 24 wherein said at least one of said first or second measurements measures a spectrally filtered emission signal.
